(19) United States
(12) Patent Application Publication
(10) Pub. No.: US 2022/0409598 A1
(43) Pub. Date: Dec. 29, 2022

(54) METHOD OF CONTROLLING BLOOD SUGAR LEVEL AND TREATMENT OF DIABETES AND RELATED CONDITIONS

(71) Applicant: ILDONG PHARMACEUTICAL CO., LTD., Seoul (KR)

(72) Inventors: Jong Min YOON, Hwaseong-si (KR); Don-Gil Lee, Hwaseong-si (KR); In-Gyu Je, Hwaseong-si (KR); YoonSung Song, Hwaseong-si (KR); JunGu Park, Hwaseong-si (KR); Soo Yong Chung, Seongnam-si (KR); Jung Ho Kim, Seongnam-si (KR); Yearin Jun, Seongnam-si (KR)

(73) Assignee: ILDONG PHARMACEUTICAL CO., LTD., Seoul (KR)

(21) Appl. No.: 17/843,587

(22) Filed: Jun. 17, 2022

Related U.S. Application Data
(60) Provisional application No. 63/212,853, filed on Jun. 21, 2021.

(51) Int. Cl. A61K 31/443; A61P 3/10; A61K 45/06

(52) U.S. Cl. CPC A61K 31/443; A61P 3/10; A61K 45/06; A61K 9/0053

(57) ABSTRACT

Methods of treating a subject with diabetes or pre-diabetes include administering an effective amount of a phenyl propionic acid of the Formula (I), an isomer, or a pharmaceutically acceptable salt thereof to the subject to lower one or more of HbA1c level, fasting plasma glucose level, 2-hour oral glucose tolerance test (OGTT) result level, and random plasma glucose level.

### CROSS REFERENCE TO RELATED APPLICATIONS

This application claims benefit of and right of priority based on U.S. Provisional Application No. 63/212,853 filed Jun. 21, 2021, of which content is incorporated by reference in its entirety.

### FIELD

A method of controlling blood sugar level and treating diabetes and related conditions is provided.

### BACKGROUND

According to World Health Organization (WHO), diabetes is a chronic, metabolic disease characterized by elevated levels of blood glucose (or blood sugar), which leads over time to serious damage to the heart, blood vessels, eyes, kidneys and nerves. Diabetes occurs either when the pancreas does not produce enough insulin or when the body cannot effectively use the insulin it produces. Insulin is a hormone that regulates blood sugar. Hyperglycemia or raised blood sugar is a common effect of uncontrolled diabetes and over time leads to serious damage to many of the body's systems, especially the nerves and blood vessels.

Insulin regulates the metabolism of carbohydrates, fats and protein by promoting the absorption of glucose from the blood into fat, liver and skeletal muscle cells. Pancreatic beta cells (β cells) are sensitive to glucose concentrations in the blood. In non-diabetics, when glucose concentrations in the blood are high, the pancreatic beta cells secrete insulin into the blood; when glucose levels are low, secretion of insulin is inhibited. Pancreatic alpha cells secrete glucagon, another peptide hormone, into the blood to raise the concentration of glucose in the blood in the opposite manner, i.e., increased secretion when blood glucose is low, and decreased secretion when glucose concentrations are high. The secretion of insulin and glucagon into the blood in response to the blood glucose concentration is the primary mechanism responsible for keeping the glucose levels in the extracellular fluids within narrow limits.

The WHO reports the trend of increasing the number of diabetes and premature mortality from diabetes. For example, between 2000 and 2016, there was a 5% increase in premature mortality from diabetes. According to the WHO, the number of people with diabetes has risen from 108 million in 1980 to 422 million in 2014. In 2014, 8.5% of adults aged 18 years and older had diabetes. In 2012 diabetes was the direct cause of 1.5 million deaths and high blood glucose was the cause of another 2.2 million deaths. Diabetes is a major cause of blindness, kidney failure, heart attacks, stroke and lower limb amputation. Adults with diabetes have a 2-3-fold increased risk of heart attacks and strokes. Combined with reduced blood flow, neuropathy (nerve damage) in the feet increases the chance of foot ulcers, infection and eventual need for limb amputation. Diabetes is among the leading causes of kidney failure.

Diabetes or diabetes mellitus (DM) is mainly divided into Type 1 and Type 2 diabetes. Type 2 diabetes (formerly called non-insulin-dependent, or adult-onset) (T2DM) results from the body's ineffective use of insulin. The majority of people with diabetes have type 2 diabetes. Dietary habits, lack of exercise and irregular lifestyle have been pointed out as the indirect causes of such increase in the occurrence of type 2 diabetes. Until recently, this type of diabetes was seen only in adults but it is now also occurring increasingly frequently in children. Type 1 diabetes (previously known as insulin-dependent, juvenile or childhood-onset) (T1DM) is characterized by deficient insulin production and requires daily administration of insulin. Neither the cause of Type 1 diabetes nor the means to prevent it are known. The third group of diabetes is gestational diabetes that is Hyperglycemia with blood glucose values above normal but below those diagnostic of diabetes. Gestational diabetes occurs during pregnancy.

According to the guidelines of the American Diabetes Association criteria for diabetes diagnosis involves four options: a fasting plasma glucose level greater than or equal to 126 mg/dl, a 2-hour oral glucose tolerance test (OGTT) providing a plasma glucose value of greater than or equal to 200 mg/dl, an HbA1c value greater than or equal to 6.5%, or a random plasma glucose level greater than or equal to 200 mg/dl in individuals with symptoms of hyperglycemia or hyperglycemic crisis. Pre-diabetes is defined as having a fasting glucose level of between 100 mg/dl and 125 mg/dl, a 2-hour OGTT plasma glucose level of between 140 mg/dl and 199 mg/dl, or an HbA1c value between 5.7 and 6.4%. Pre-diabetes may be considered to be a major risk factor for the development of type 2 diabetes mellitus, cardiovascular disease and mortality.

In the monitoring of the treatment of diabetes, the HbA1c value, which is the product of a non-enzymatic glycation of the hemoglobin B chain, may be considered to be an important parameter. HbA1c values depend on the blood sugar level and the life time of erythrocytes in the blood. HbA1c values typically reflect the average blood sugar level 4-12 weeks prior to removal and testing of a patient's blood. Diabetic patients whose HbA1c level has been well controlled over a long time treatment (i.e. <6.5% of the total hemoglobin in the sample) are typically better protected from diabetic microangiopathy. The available treatments for diabetes can give the diabetic an average improvement in their HbA1c level of the order of 1.0-1.5%. However, this reduction in the HbA1C level may not be sufficient in all diabetics to bring them into the desired target range of <7.0%, preferably <6.5%, more preferably <6% HbA1c, and even more preferably <6% HbA1c.

In addition to improvement of the HbA1c level, other recommended therapeutic goals for type 2 diabetes patients are improvement of fasting plasma glucose (FPG) and of postprandial plasma glucose (PPG) levels to normal or as near normal as possible. Desired target ranges of fasting plasma glucose can be, e.g., 90-130 mg/dL or <110 mg/dL, and of two-hour postprandial plasma glucose can be, e.g., <180 mg/dL or <140 mg/dL.

Diet therapy and exercise therapy are frequently considered to be essential in the treatment of diabetes. When these therapies do not sufficiently control the condition of patients (especially their blood sugar levels), an oral or non-oral antidiabetic agent can be used for the treatment of diabetes. Conventional antidiabetic or hypoglycemic agents include, without limitation, biguanides, dipeptidyl peptidase-4 (DPP-4) inhibitors, sulphonylureas, thiazolidinediones, meglitinides (aka glinides), alpha-glucosidase blockers, GLP-1 and GLP-1 analogs, as well as insulin and insulin analogs.

However, these conventional drugs have shortcomings that cannot be overcome. For example, metformin of biguanide type, the primary treatment for type 2 diabetes, places patients at risk of diarrhea, abdominalgia, dyspepsia, and lack of durability in long-term use. Sulfonylureas, independent from blood glucose level, stimulate pancreatic β-cells and thus place patients at risk of hypoglycemia. Liver safety concerns, cardiovascular risks, weight gain, and risk of bladder cancer have been reported with thiazolidinediones, so the drug has been withdrawn from the market. Sodium-glucose co-transporter-2 (SGLT-2) inhibitors make patients become vulnerable to urinary tract and genital infections, and α-glucosidase inhibitors may induce side-effects including dyspepsia and diarrhea. Furthermore, dipeptidyl peptidase-4 (DPP-4) inhibitors are limited to patients without any renal conditions. GLP-1 or GLP-1 analogues can be associated with gastrointestinal adverse effects such as dyspepsia, flatulence or diarrhea, or nausea or vomiting.

Therefore, there is a need for improved therapy in treatment of diabetes and/or diabetes related metabolic diseases.

### SUMMARY

Methods of treating a metabolic disease described herein include administering a phenyl propionic acid of the Formula (I), an isomer, or a pharmaceutically acceptable salt thereof to a subject in need of lowering one or more of HbA1c level, fasting plasma glucose level, 2-hour oral glucose tolerance test (OGTT) result level, and random plasma glucose level:

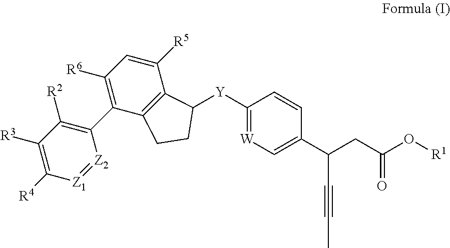

R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ and R⁴ are each independently hydrogen, halogen, cyano, C₁₋₄ linear or branched alkoxy, or OR⁸;
wherein R⁸ is hydrogen, C₃₋₁₀ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ a linear or branched alkoxy;
Y is NH or O;
Z₁, Z₂ and W are each independently CR⁷ or N;
wherein R⁷ is hydrogen, halogen, cyano, hydroxyl, C₁₋₄ linear or branched alkyl, or C₁₋₄ linear or branched alkoxy.

In embodiments, an effective amount of a phenyl propionic acid of the Formula (I), an isomer, or a pharmaceutically acceptable salt thereof, or a pharmaceutical composition comprising the above is administered to a subject in need thereof to treat a metabolic disease. In embodiments, the metabolic disease is diabetes. In embodiments the diabetes is type 2 diabetes. In embodiments the diabetes is type 1 diabetes. In embodiments, the metabolic disease is pre-diabetes. In embodiments, the HbA1c level is lowered by an amount greater than 0.25% compared to that of prior to the treatment. In embodiments, the HbA1c level is lowered by an amount greater than 0.5%. In embodiments, the HbA1c level is lowered by an amount greater than 0.75%. In embodiments, the HbA1c level is lowered by an amount greater than 1.0%. In embodiments, the HbA1c level is lowered by an amount greater than 1.5%. In embodiments, the HbA1c level is lowered by an amount greater than 2.0%. In embodiments, a reduction in the range from 0.25% to 3% can be achieved. In embodiments, the HbA1c level is lowered by an amount greater than 1.0%. In embodiments, the HbA1c level is lowered by an amount greater than 1.5%.

In embodiments, the compound of Formula (I), an isomer, or a pharmaceutically acceptable salt thereof may be administered in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, insulin and an insulin analog. In embodiments, methods of treating a subject with diabetes include administering the compound of Formula (I), an isomer, or a pharmaceutically acceptable salt thereof to the subject to lower one or more of HbA1c level, fasting plasma glucose level, 2-hour oral glucose tolerance test (OGTT) result level, and random plasma glucose level. In embodiments, the diabetes is type 2 diabetes. In embodiments, the diabetes is type 1 diabetes. In embodiments, the HbA1c level is lowered by an amount greater than 0.25% compared to that of prior to the treatment. In embodiments, the HbA1c level is lowered by an amount greater than 0.5%. In embodiments, the HbA1c level is lowered by an amount greater than 0.75%. In embodiments, the HbA1c level is lowered by an amount greater than 1.0%. In embodiments, the HbA1c level is lowered by an amount greater than 1.5%. In embodiments, the HbA1c level is lowered by an amount greater than 2.0%. In embodiments, a reduction in the range from 0.25% to 3% can be achieved. In embodiments, the HbA1c level is lowered by an amount greater than 1.0%. In embodiments, the HbA1c level is lowered by an amount greater than 1.5%. In embodiments, the compound of Formula (I), an isomer, or a pharmaceutically acceptable salt thereof may be administered in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, insulin and an insulin analog.

In another embodiments, provided is the use of the compound of Formula (I), an isomer, or a pharmaceutically acceptable salt thereof in manufacturing a medicament for treating a subject in need thereof to lower one or more of HbA1c level, fasting plasma glucose level, 2-hour oral glucose tolerance test (OGTT) result level, and random plasma glucose level. In embodiments, the subject may suffer from a metabolic disease. In embodiments, the metabolic disease may be diabetes or pre-diabetes. In embodiments, the diabetes is type 2 diabetes. In embodiments, the diabetes is type 1 diabetes. In embodiments, the HbA1c level is lowered by an amount greater than 0.25% compared to that of prior to the treatment. In embodiments, the HbA1c level is lowered by an amount greater than 0.5%. In embodiments, the HbA1c level is lowered by an amount greater than 0.75%. In embodiments, the HbA1c level is lowered by an amount greater than 1.0%. In embodiments, the HbA1c level is lowered by an amount greater than 1.5%. In embodiments, the HbA1c level is lowered by an amount greater than 2.0%. In embodiments, a reduction in the range from 0.25% to 3% can be achieved. In embodiments, the HbA1c level is lowered by an amount greater than 1.0%. In embodiments, the HbA1c level is lowered by an amount greater than 1.5%. In embodiments, the medicament may be administered in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, insulin and an insulin analog.

In still another embodiments, a pharmaceutical composition for treating a subject with a metabolic disease comprises, as an active ingredient, the compound of Formula (I), an isomer, or a pharmaceutically acceptable salt thereof, wherein the compound of Formula (I), an isomer, or a pharmaceutically acceptable salt thereof lowers one or more of HbA1c level, fasting plasma glucose level, 2-hour oral glucose tolerance test (OGTT) result level, and random plasma glucose level. In embodiments, the metabolic disease may be diabetes or pre-diabetes. In embodiments, the diabetes is type 2 diabetes. In embodiments, the diabetes is type 1 diabetes. In embodiments, the HbA1c level is lowered by an amount greater than 0.25% compared to that of prior to the treatment. In embodiments, the HbA1c level is lowered by an amount greater than 0.5%. In embodiments, the HbA1c level is lowered by an amount greater than 0.75%. In embodiments, the HbA1c level is lowered by an amount greater than 1.0%. In embodiments, the HbA1c level is lowered by an amount greater than 1.5%. In embodiments, the HbA1c level is lowered by an amount greater than 2.0%. In embodiments, a reduction in the range from 0.25% to 3% can be achieved. In embodiments, the pharmaceutical composition may be administered in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, insulin and an insulin analog. In embodiments, the pharmaceutical composition may include one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, insulin and an insulin analog, in a same formulation with the compound of Formula (I), an isomer, or a pharmaceutically acceptable salt thereof or in a different formulation. When the compound of Formula (I) an isomer, or a pharmaceutically acceptable salt thereof is contained in a different formulation from a formulation including one or more hypoglycemic agents, they may be administered simultaneously or separately.

In still another embodiments, the method is
(i) preventing, slowing the progression of, delaying, or treating a metabolic disorder selected from the group consisting of type 1 diabetes, type 2 diabetes, impaired glucose tolerance, impaired fasting blood glucose, hyperglycemia, postprandial hyperglycemia, overweight, obesity, and metabolic syndrome,
(ii) improving glycemic control and/or for reducing of fasting plasma glucose and/or of postprandial plasma glucose and/or of glycosylated hemoglobin HbA1c,
(iii) preventing, slowing, delaying, or reversing progression from impaired glucose tolerance, insulin resistance, and/or from metabolic syndrome to type 2 diabetes mellitus,
(iv) preventing, slowing the progression of, delaying, or treating of a condition or disorder selected from the group consisting of cataracts, nephropathy, retinopathy, neuropathy, learning and memory impairment, neurodegenerative or cognitive disorders, cardio- or cerebrovascular diseases, tissue ischemia, diabetic foot ulcer, arteriosclerosis, hypertension, endothelial dysfunction, myocardial infarction, acute coronary syndrome, unstable angina pectoris, stable angina pectoris, stroke, peripheral arterial occlusive disease, cardiomyopathy, heart failure, heart rhythm disorders, and vascular restenosis,
(v) reducing body weight and/or body fat or preventing an increase in body weight and/or body fat or facilitating a reduction in body weight and/or body fat,
(vi) preventing, slowing, delaying or treating the degeneration of pancreatic beta cells and/or the decline of the functionality of pancreatic beta cells and/or for improving and/or restoring or protecting the functionality of pancreatic beta cells and/or restoring the functionality of pancreatic insulin secretion,
(vii) preventing, slowing, delaying or treating diseases or conditions attributed to an abnormal accumulation of liver or ectopic fat,
(viii) maintaining and/or improving the insulin sensitivity and/or for treating or preventing hyperinsulinemia and/or insulin resistance,
(ix) preventing, slowing progression of, delaying, or treating new onset diabetes after transplantation (NODAT) and/or post-transplant metabolic syndrome (PTMS),
(x) preventing, delaying, or reducing NODAT and/or PTMS associated complications including micro- and macrovascular diseases and events, graft rejection, infection, and death, or
(xi) treating hyperuricemia and hyperuricemia associated conditions in a subject in need thereof,
the method comprising administering to the subject an effective amount of:
(a) the compound of Formula (I), an isomer, or a pharmaceutically acceptable salt thereof,
(b) optionally, a second hypoglycemic agent selected from the group consisting of biguanides, thiazolidinediones, sulfonylureas, glinides, alpha-glucosidase blockers, GLP-1 and GLP-1 analogues, or a pharmaceutically acceptable salt thereof, and,
(c) optionally, a third hypoglycemic agent different from (b) and selected from the group consisting of biguanides, thiazolidinediones, sulfonylureas, glinides, alpha-glucosidase blockers, GLP-1 and GLP-1 analogues, or a pharmaceutically acceptable salt thereof.

In the above methods, use, and pharmaceutical compositions, the compound of Formula (I) may be
(1) (S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid;
(2) (S)-3-(4-(((R)-7-fluoro-4-(6-((3-methyloxetan-3-yl)methoxy)pyridin-3-yl)-2,3-dihydro-1H-inden-1-yl)oxy)phenyl)hex-4-ynoic acid;
(3) (S)-3-(4-(((R)-4-(6-(2-(1,1-dioxidothiomorpholino)ethoxy)pyridin-3-yl)-7-fluoro-2,3-dihydro-1H-inden-1-yl)oxy)phenyl)hex-4-ynoic acid;
(4) (S)-3-(4-(((R)-7-fluoro-4-(6-(oxetan-3-yloxy)pyridin-3-yl)-2,3-dihydro-1H-inden-1-yl)oxy)phenyl)hex-4-ynoic acid;
(5) (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid;
(6) (S)-3-(4-(((R)-7-fluoro-4-(6-((tetrahydro-2H-pyran-4-yl)oxy)pyridin-3-yl)-2,3-dihydro-1H-inden-1-yl)oxy)phenyl)hex-4-ynoic acid;
(7) (S)-3-(4-(((R)-7-fluoro-4-(6-(((S)-tetrahydrofuran-3-yl)oxy)pyridin-3-yl)-2,3-dihydro-1H-inden-1-yl)oxy)phenyl)hex-4-ynoic acid;
(8) (S)-3-(4-(((R)-7-fluoro-4-(4-methyl-6-(((R)-tetrahydrofuran-3-yl)oxy)pyridin-3-yl)-2,3-dihydro-1H-inden-1-yl)oxy)phenyl)hex-4-ynoic acid;
(9) (S)-3-(4-(((R)-7-fluoro-4-(2-methyl-6-(((R)-tetrahydrofuran-3-yl)oxy)pyridin-3-yl)-2,3-dihydro-1H-inden-1-yl)oxy)phenyl)hex-4-ynoic acid;
(10) (S)-3-(4-(((R)-4-(5-chloro-6-(((R)-tetrahydrofuran-3-yl)oxy)pyridin-3-yl)-7-fluoro-2,3-dihydro-1H-inden-1-yl)oxy)phenyl)hex-4-ynoic acid;
(11) (S)-3-(4-(((R)-7-fluoro-4-(5-(((R)-tetrahydrofuran-3-yl)oxy)pyridin-2-yl)-2,3-dihydro-1H-inden-1-yl)oxy)phenyl)hex-4-ynoic acid;
(12) (S)-3-(4-(((R)-7-fluoro-4-(4-methyl-6-((3-methyloxetan-3-yl)methoxy)pyridin-3-yl)-2,3-dihydro-1H-inden-1-yl)oxy)phenyl)hex-4-ynoic acid;
(13) (S)-3-(4-(((R)-7-fluoro-4-(2-methyl-6-((3-methyloxetan-3-yl)methoxy)pyridin-3-yl)-2,3-dihydro-1H-inden-1-yl)oxy)phenyl)hex-4-ynoic acid;
(14) (S)-3-(4-(((R)-7-fluoro-4-(5-((3-methyloxetan-3-yl)methoxy)pyridin-2-yl)-2,3-dihydro-1H-inden-1-yl)oxy)phenyl)hex-4-ynoic acid;
(15) (S)-3-(4-(((R)-7-fluoro-4-(5-(((R)-tetrahydrofuran-3-yl)oxy)pyridin-3-yl)-2,3-dihydro-1H-inden-1-yl)oxy)phenyl)hex-4-ynoic acid;
(16) (S)-3-(4-(((R)-7-fluoro-4-(5-((tetrahydro-2H-pyran-4-yl)oxy)pyridin-3-yl)-2,3-dihydro-1H-inden-1-yl)oxy)phenyl)hex-4-ynoic acid;
(17) (S)-3-(4-(((R)-4-(5-chloro-6-((tetrahydro-2H-pyran-4-yl)oxy)pyridin-3-yl)-7-fluoro-2,3-dihydro-1H-inden-1-yl)oxy)phenyl)hex-4-ynoic acid;
(18) (S)-3-(4-(((R)-4-(5-cyano-6-(((R)-tetrahydrofuran-3-yl)oxy)pyridin-3-yl)-7-fluoro-2,3-dihydro-1H-inden-1-yl)oxy)phenyl)hex-4-ynoic acid;
(19) (S)-3-(4-(((R)-4-(5-cyano-6-((tetrahydro-2H-pyran-4-yl)oxy)pyridin-3-yl)-7-fluoro-2,3-dihydro-1H-inden-1-yl)oxy)phenyl)hex-4-ynoic acid;
(20) (S)-3-(4-(((R)-5-cyano-4-(6-(((R)-tetrahydrofuran-3-yl)oxy)pyridin-3-yl)-2,3-dihydro-1H-inden-1-yl)oxy)phenyl)hex-4-ynoic acid;
(21) (S)-3-(4-(((R)-5-fluoro-4-(6-(((R)-tetrahydrofuran-3-yl)oxy)pyridin-3-yl)-2,3-dihydro-1H-inden-1-yl)oxy)phenyl)hex-4-ynoic acid;
(22) (S)-3-(4-(((R)-5-methoxy-4-(6-(((R)-tetrahydrofuran-3-yl)oxy)pyridin-3-yl)-2,3-dihydro-1H-inden-1-yl)oxy)phenyl)hex-4-ynoic acid;
(23) (S)-3-(4-(((R)-5-cyano-4-(6-((tetrahydro-2H-pyran-4-yl)oxy)pyridin-3-yl)-2,3-dihydro-1H-inden-1-yl)oxy)phenyl)hex-4-ynoic acid;
(24) (S)-3-(4-(((R)-5-fluoro-4-(6-((tetrahydro-2H-pyran-4-yl)oxy)pyridin-3-yl)-2,3-dihydro-1H-inden-1-yl)oxy)phenyl)hex-4-ynoic acid;
(25) (S)-3-(4-(((R)-5-methoxy-4-(6-((tetrahydro-2H-pyran-4-yl)oxy)pyridin-3-yl)-2,3-dihydro-1H-inden-1-yl)oxy)phenyl)hex-4-ynoic acid;
(26) (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid; or
(27) 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid.

In the above methods, uses, and/or compositions, the subject shows one, two, or more of the following conditions:
(a) a fasting blood glucose or serum glucose concentration greater than 100 mg/dL or greater than 110 mg/dL, in particular greater than 125 mg/dL;
(b) a postprandial plasma glucose equal to or greater than 140 mg/dL;
(c) an HbA1c value equal to or greater than 5.7%, in particular equal to or greater than 6.5%, in more particular equal to or greater than 7.0%, especially equal to or greater than 7.5%, even more particularly equal to or greater than 8.0%.

### BRIEF DESCRIPTION OF DRAWINGS

FIG. 1 summarizes the animals, doses, routes, and frequencies of administering the compound of Reference Example 1 to obtain pharmacokinetic and pharmarcodynamic data (PK/PD).

FIG. 2 shows simulated human PK parameters of the compound of Reference Example 1.

FIG. 3 is a simulated glucose concentration-time profiles in human after the compound of Reference Example 1 and glucose administration.

FIG. 4A and FIG. 4B are graphs showing mean plasma concentration of Reference Example 1 compound following single dose of 0.5 mg, 1 mg, 2 mg, 5 mg, and 10 mg in linear scale and semi-logarithmic scale, respectively.

FIG. 5A and FIG. 5B are graphs showing mean plasma concentration of Reference Example 1 compound following single dose of 0.5 mg (fasted), 1 mg (fasted), 2 mg (fasted), 5 mg (fasted), and 5 mg (fed) in linear scale and semi-logarithmic scale, respectively.

FIG. 6 shows the results of the exposure and efficacy of Reference Example 1 compound (at 0.1 mg/kg, 0.3 mg/kg, and 1 mg/kg doses) in SD rats and the predicted exposure at MAD doses in human.

FIG. 7 shows the accumulation of glycocholic acid (GCA) by Fasiglifam, Troglitazone, Pioglitazone, and Reference Example 1 compound at various concentrations.

FIG. 8 shows inhibitory effects of fasiglifam and Reference Example 1 compound on mitochondrial function, evaluated using HepaRG cell.

FIG. 9 shows the HμRELTOX™ assay results obtained for fasiglifam and Reference Example 1 compound.

FIG. 10 and FIG. 11 show the effects of fasiglifam and Reference Example 1 compound on various hepatic transcription factors at various concentrations.

FIG. 12 summarizes the DILI Assessment comparing the Reference Example 1 compound and fasiglifam.

### DETAILED DESCRIPTION

Provided herein are methods and compositions for use in treating metabolic disorders such as diabetes, including type 1 diabetes, type 2 diabetes and pre-diabetes. In embodiments, administration of the compound of Formula (I), an isomer, or a pharmaceutically acceptable salt thereof, alone or optionally in combination with the one or more hypoglycemic agents (i.e., optional the second active ingredient and optionally the third active ingredient) such as a biguanide, a dipeptidyl peptidase-4 (DPP-4) inhibitor, a sulphonylurea, a thiazolidinedione, a meglitinide (glinide), an alpha-glucosidase blocker, a glucagon-like peptide-1 receptor agonist, insulin or an insulin analog can reduce symptoms of, prevent, slow the progression of, or delay a metabolic disorder such as, e.g., type 1 diabetes, type 2 diabetes, pre-diabetes, impaired glucose tolerance (IGT), impaired fasting blood glucose (IFG), hyperglycemia, and postprandial hyperglycemia.

Methods and compositions for use in treating metabolic disorders disclosed herein are used for improving glycemic control. “Improvement of glycemic control”, “improving glycemic control” or “glycemic control” refers to improvement of glucose tolerance, improvement of postprandial plasma glucose concentration, improvement of fasting plasma glucose concentration, improvement of the HbA1c value or/and improvement of fasting plasma insulin concentration.

Methods and compositions for use in treating metabolic disorders disclosed herein improve, reduce or alleviate symptoms or conditions associated with metabolic diseases. Conditions associated with metabolic disorders can include, e.g., sleep apnea, obesity, dyslipidemia, hyperlipidemia, hypercholesterolemia, hypertension, atherosclerosis, endothelial dysfunction, osteoporosis, chronic systemic inflammation, non-alcoholic fatty liver disease (NAFLD), retinopathy, neuropathy, nephropathy and/or metabolic syndrome. Methods and compositions for use in treating metabolic disorders disclosed herein can improve glycemic control, e.g., reduce fasting plasma glucose, reduce postprandial plasma glucose and/or reduce HbA1c. Methods and compositions for use in treating metabolic disorders disclosed herein can prevent, slow, delay or reverse progression from impaired glucose tolerance (IGT), impaired fasting blood glucose (IFG), insulin resistance from metabolic syndrome, to type 2 diabetes.

Methods and compositions for use in treating metabolic disorders disclosed herein can prevent, reduce the risk, slow the progression, delay or treat complications of diabetes such as micro- and macrovascular diseases including nephropathy, micro- or macroalbuminuria, proteinuria, retinopathy, cataracts, neuropathy, learning or memory impairment, neurodegenerative or cognitive disorders, cardio- or cerebrovascular diseases, tissue ischemia, diabetic foot ulcers, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke. Methods and compositions for use in treating metabolic disorders disclosed herein can prevent, slow the progression of, delay or treat type 2 diabetes with primary or secondary failure to conventional (oral) hypoglycemic mono- or combination therapy. Methods and compositions for use in treating metabolic disorders disclosed herein can achieve a reduction in the dose of conventional hypoglycemic medication required for adequate therapeutic effect, thereby reducing the risk for adverse effects associated with conventional hypoglycemic medication. Methods and compositions for use in treating metabolic disorders disclosed herein can maintain and/or improve insulin sensitivity and/or treat or prevent hyperinsulinemia and/or insulin resistance.

### DEFINITION OF TERMS

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The term “significant” or “significantly” refers to statistical significance and generally means a two standard deviation (2SD) or greater difference.

The term “baseline” means the values of glucose level, HbA1c level, fasting plasma glucose level, 2-hour oral glucose tolerance test (OGTT) result level, and random plasma glucose level at day 1 for the studies described in EXAMPLES.

The term “active ingredient” of a pharmaceutical composition according to the embodiments means the compound of Formula (I), an isomer, or a pharmaceutically acceptable salt thereof, and/or the second hypoglycemic agent, and/or the third hypoglycemic agent described herein.

The term “euglycemia” is defined as the condition in which a subject has a fasting blood glucose concentration within the normal range, greater than 70 mg/dL (3.89 mmol/L) and less than 110 mg/dL (6.11 mmol/L) or 100 mg/dL (5.6 mmol/L). The word “fasting” has the usual meaning as a medical term.

The term “hyperglycemia” is defined as the condition in which a subject has a fasting blood glucose concentration above the normal range, greater than 110 mg/dL (6.11 mmol/L) or 100 mg mg/dL (5.6 mmol/L). The word “fasting” has the usual meaning as a medical term.

The term “hypoglycemia” is defined as the condition in which a subject has a blood glucose concentration below the normal range of 60 to 115 mg/dL (3.3 to 6.3 mmol/L), in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/1), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/I.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio <1.0 (for men) or <0.8 (for women).

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference 85 cm in men and 90 cm in women (see e.g. investigating committee for the diagnosis of metabolic syndrome in Japan).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycemic-hyperinsulinemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

Insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52 (Suppl. 1): A459) and to a euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9:921-8):

HOMA-IR=[fasting serum insulin(μU/mL)]×[fasting plasma glucose(mmol/L)/22.5].

Other parameters such as the patient's triglyceride concentration can be used as an additional indicator. For example, as increased triglyceride levels correlate significantly with the presence of insulin resistance.

Patients with a predisposition for the development of IGT or IFG or type 2 diabetes are those having euglycemia with hyperinsulinemia and are by definition, insulin resistant. If insulin resistance can be detected, this is a particularly strong indication of the presence of pre-diabetes. Thus, it may be that in order to maintain glucose homoeostasis a person needs 2-3 times as much insulin as a healthy person, without any clinical symptoms.

The term “pre-diabetes” is the condition wherein an individual is pre-disposed to the development of type 2 diabetes. Pre-diabetes extends the definition of impaired glucose tolerance to include individuals with a fasting blood glucose within the high normal range 100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

Insulin resistance is defined as the clinical condition in which an individual has a HOMA-IR score >4.0 or a HOMA-IR score above the upper limit of normal as defined for the laboratory performing the glucose and insulin assays.

The term “type 2 diabetes” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients with type 2 diabetes and failed with a secondary antidiabetic drug, and who show indication for insulin therapy and progression to micro- and macrovascular complications e.g. diabetic nephropathy, or coronary heart disease (CHD).

The term “HbA1c” refers to the product of a non-enzymatic glycation of the hemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (for example, <6.0% of the total hemoglobin in the sample), are significantly better protected against diabetic microangiopathy. For example, metformin on its own achieves an average improvement in the HbA1c value in the diabetic of the order of 1.0-1.5%. This reduction of the HbA1c value is not sufficient in all diabetics to achieve the desired target range of <6.5%, preferably <6%, and more preferably <5.7% HbA1c.

The term “insufficient glycemic control” or “inadequate glycemic control” in the scope of the present invention means a condition wherein patients show HbA1c values above 5.7%, in particular 6.5%, in more particular above 7.0%, even more preferably above 7.5%, especially above 8%.

The “metabolic syndrome” is a syndrome complex with the cardinal feature being insulin resistance. According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome may be made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference ≥85 cm in men and ≥90 cm in women;
2. Triglycerides: ≥150 mg/dL
3. HDL-cholesterol <40 mg/dL in men
4. Blood pressure ≥130/85 mm Hg (SBP≥130 or DBP≥85)
5. Fasting blood glucose ≥110 mg/dL or ≥100 mg/dL.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The term “hyperuricemia” denotes a condition of high serum total urate levels. In human blood, uric acid concentrations between 3.6 mg/dL (ca. 214 μmol/L) and 8.3 mg/dL (ca. 494 μmol/L) are considered normal by the American Medical Association. High serum total urate levels, or hyperuricemia, are often associated with several maladies. For example, high serum total urate levels can lead to a type of arthritis in the joints known as gout. Gout is a condition created by a build up of monosodium urate or uric acid crystals on the articular cartilage of joints, tendons and surrounding tissues due to elevated concentrations of total urate levels in the blood stream. The build up of urate or uric acid on these tissues provokes an inflammatory reaction of these tissues. Saturation levels of uric acid in urine may result in kidney stone formation when the uric acid or urate crystallizes in the kidney. Additionally, high serum total urate levels are often associated with the so-called metabolic syndrome, including cardiovascular disease and hypertension.

An “effective amount” of a polynucleotide encoding a fusion protein as disclosed herein is an amount sufficient to carry out a specifically stated purpose. An “effective amount” can be determined empirically and in a routine manner, in relation to the stated purpose.

Terms such as “treating” or “treatment” or “to treat” or “alleviating” or “to alleviate” refer to therapeutic measures that cure, slow down, lessen symptoms of, halt progression of a diagnosed pathologic condition or disorder, Thus, the subjects in need of treatment include those already diagnosed with or suspected of having the disorder.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the subject is a human.

“Pharmaceutically acceptable” refers to molecular entities and compositions that are “generally regarded as safe,” e.g., they are biologically or pharmacologically compatible for in vivo use in animals or humans, which are physiologically tolerable and do not typically produce an allergic or similar untoward reaction, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

The term “pharmaceutically acceptable salt”, as used herein, refers to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

“Co-administered with,” “in combination with,” “administered in combination with,” “a combination of,” or “administered along with” may be used interchangeably and mean that two or more agents are administered in the course of therapy. The agents may be administered together at the same time or separately in spaced apart intervals. The agents may be administered in a single dosage form or in separate dosage forms.

As used herein, “sustained release” or “extended release” means that the release of the therapeutically active agent occurs over an extended period of time leading to lower peak plasma concentrations and/or is directed to a prolonged Tₘₐₓ as compared to “conventional release” or “immediate release.” For example, extended release compositions may have a mean Tₘₐₓ of about 5 or more hours.

### Compound of Formula (I)

The active ingredient of the methods and compositions according to embodiments is a compound of Formula (I), an isomer, or a pharmaceutically acceptable salt thereof:

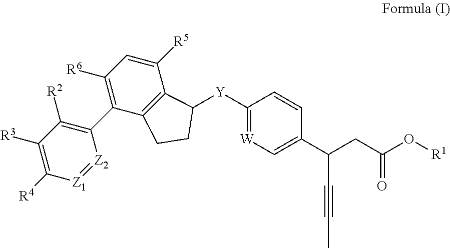

R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ and R⁴ are each independently hydrogen, halogen, cyano, C₁₋₄ linear or branched alkoxy, or OR⁸;
wherein R⁸ is hydrogen, C₃₋₁₀ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ linear or branched alkoxy;
Y is NH or O;
Z₁, Z₂ and W are each independently CR⁷ or N;
wherein R⁷ is hydrogen, halogen, cyano, hydroxyl, C₁₋₄ linear or branched alkyl, or C₁₋₄ linear or branched alkoxy.

The compound of Formula (I) is a G-protein coupled receptor 40 (GPR40) agonist.

GPR40 is a seven-transmembrane protein, a type of G-protein-coupled receptor (GPCR) of the rhodopsin family, and is primarily expressed in β-cells of pancreatic islets. Since its primary ligands are medium-to-long change fatty acids, the receptor is also known as free fatty acid receptor 1 (FFAR1).

The mechanism of pancreatic β-cell's insulin secretion through GPR40 is mainly determined by either ligands or GPR40 agonists that bind to the receptor. When binding activates the receptor, primary signaling pathway for insulin secretion is promoted through Gαq/11, which is a type of subunits of GPCR. Then, the pathway hydrolyzes cell membrane phospholipids through phospholipase C (PLC) to produce diacylglycerol (DAG) and inositol trisphosphate (IP3), which subsequently activate protein kinase D1 (PKD1) to induce F-actin protein modification, and calcium ion secretion to ultimately induce insulin secretion.

The mechanism that GPR40 activation induces insulin secretion with blood glucose-dependent manner was proven through experiments using rodent models. (Diabetes, 2007, 56, 1087-1094: Diabetes, 2009, 58, 1067-1076). Such blood glucose-dependent mechanism of insulin secretion has no risk of hypothermia, which makes GPR40 an attractive target for novel drug development. In addition, GPR40 is involved in maintaining pancreatic β-cell survival through regulation of PIX-1 and BCL2, which also results in sustaining of efficacy even in a long-term treatment (BMC Cell Biol., 2014, 15, 24). Furthermore, since the distribution of GPR40 expression is relatively limited, there is low risk of adverse effects in other organs, and improving blood-glucose homeostasis through GPR40 activation is potentially involved in other metabolic disorders including obesity and hypertension.

Based on such advantages, for the past few years, industrial efforts have made investments in the development of GPR40 agonists, but no drug has been released to the market. Among the discoveries, Fasiglifam of Takeda, the first GPR40 agonist to enter clinical trials, has been shown its glucose-lowering efficacy in patients with T2DM in phase II trials. However, despite its efficacy, the compound was discontinued in phase Ill trial due to liver safety concerns (Diabetes obes metab., 2015, 17, 675-681).

The compound of Formula (I), its isomers, and pharmaceutically acceptable salts may be prepared by a process described in co-pending U.S. application Ser. No. 16/467,654, of which the content is incorporated by reference herein. The GPR40 agonistic activity of a compound of Formula (I), its isomers, and pharmaceutically acceptable salts may be evaluated by cell-based aequorin assay as described therein.

Exemplary compounds of Formula (I) include:
(1) (S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid;
(2) (S)-3-(4-(((R)-7-fluoro-4-(6-((3-methyloxetan-3-yl)methoxy)pyridin-3-yl)-2,3-dihydro-1H-inden-1-yl)oxy)phenyl)hex-4-ynoic acid;
(3) (S)-3-(4-(((R)-4-(6-(2-(1,1-dioxidothiomorpholino)ethoxy)pyridin-3-yl)-7-fluoro-2,3-dihydro-1H-inden-1-yl)oxy)phenyl)hex-4-ynoic acid;
(4) (S)-3-(4-(((R)-7-fluoro-4-(6-(oxetan-3-yloxy)pyridin-3-yl)-2,3-dihydro-1H-inden-1-yl)oxy)phenyl)hex-4-ynoic acid;
(5) (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid;
(6) (S)-3-(4-(((R)-7-fluoro-4-(6-((tetrahydro-2H-pyran-4-yl)oxy)pyridin-3-yl)-2,3-dihydro-1H-inden-1-yl)oxy)phenyl)hex-4-ynoic acid;
(7) (S)-3-(4-(((R)-7-fluoro-4-(6-(((S)-tetrahydrofuran-3-yl)oxy)pyridin-3-yl)-2,3-dihydro-1H-inden-1-yl)oxy)phenyl)hex-4-ynoic acid;
(8) (S)-3-(4-(((R)-7-fluoro-4-(4-methyl-6-(((R)-tetrahydrofuran-3-yl)oxy)pyridin-3-yl)-2,3-dihydro-1H-inden-1-yl)oxy)phenyl)hex-4-ynoic acid;
(9) (S)-3-(4-(((R)-7-fluoro-4-(2-methyl-6-(((R)-tetrahydrofuran-3-yl)oxy)pyridin-3-yl)-2,3-dihydro-1H-inden-1-yl)oxy)phenyl)hex-4-ynoic acid;
(10) (S)-3-(4-(((R)-4-(5-chloro-6-(((R)-tetrahydrofuran-3-yl)oxy)pyridin-3-yl)-7-fluoro-2,3-dihydro-1H-inden-1-yl)oxy)phenyl)hex-4-ynoic acid;
(11) (S)-3-(4-(((R)-7-fluoro-4-(5-(((R)-tetrahydrofuran-3-yl)oxy)pyridin-2-yl)-2,3-dihydro-1H-inden-1-yl)oxy)phenyl)hex-4-ynoic acid;
(12) (S)-3-(4-(((R)-7-fluoro-4-(4-methyl-6-((3-methyloxetan-3-yl)methoxy)pyridin-3-yl)-2,3-dihydro-1H-inden-1-yl)oxy)phenyl)hex-4-ynoic acid;
(13) (S)-3-(4-(((R)-7-fluoro-4-(2-methyl-6-((3-methyloxetan-3-yl)methoxy)pyridin-3-yl)-2,3-dihydro-1H-inden-1-yl)oxy)phenyl)hex-4-ynoic acid;
(14) (S)-3-(4-(((R)-7-fluoro-4-(5-((3-methyloxetan-3-yl)methoxy)pyridin-2-yl)-2,3-dihydro-1H-inden-1-yl)oxy)phenyl)hex-4-ynoic acid;
(15) (S)-3-(4-(((R)-7-fluoro-4-(5-(((R)-tetrahydrofuran-3-yl)oxy)pyridin-3-yl)-2,3-dihydro-1H-inden-1-yl)oxy)phenyl)hex-4-ynoic acid;
(16) (S)-3-(4-(((R)-7-fluoro-4-(5-((tetrahydro-2H-pyran-4-yl)oxy)pyridin-3-yl)-2,3-dihydro-1H-inden-1-yl)oxy)phenyl)hex-4-ynoic acid;
(17) (S)-3-(4-(((R)-4-(5-chloro-6-((tetrahydro-2H-pyran-4-yl)oxy)pyridin-3-yl)-7-fluoro-2,3-dihydro-1H-inden-1-yl)oxy)phenyl)hex-4-ynoic acid;
(18) (S)-3-(4-(((R)-4-(5-cyano-6-(((R)-tetrahydrofuran-3-yl)oxy)pyridin-3-yl)-7-fluoro-2,3-dihydro-1H-inden-1-yl)oxy)phenyl)hex-4-ynoic acid;
(19) (S)-3-(4-(((R)-4-(5-cyano-6-((tetrahydro-2H-pyran-4-yl)oxy)pyridin-3-yl)-7-fluoro-2,3-dihydro-1H-inden-1-yl)oxy)phenyl)hex-4-ynoic acid;
(20) (S)-3-(4-(((R)-5-cyano-4-(6-(((R)-tetrahydrofuran-3-yl)oxy)pyridin-3-yl)-2,3-dihydro-1H-inden-1-yl)oxy)phenyl)hex-4-ynoic acid;
(21) (S)-3-(4-(((R)-5-fluoro-4-(6-(((R)-tetrahydrofuran-3-yl)oxy)pyridin-3-yl)-2,3-dihydro-1H-inden-1-yl)oxy)phenyl)hex-4-ynoic acid;
(22) (S)-3-(4-(((R)-5-methoxy-4-(6-(((R)-tetrahydrofuran-3-yl)oxy)pyridin-3-yl)-2,3-dihydro-1H-inden-1-yl)oxy)phenyl)hex-4-ynoic acid;
(23) (S)-3-(4-(((R)-5-cyano-4-(6-((tetrahydro-2H-pyran-4-yl)oxy)pyridin-3-yl)-2,3-dihydro-1H-inden-1-yl)oxy)phenyl)hex-4-ynoic acid;
(24) (S)-3-(4-(((R)-5-fluoro-4-(6-((tetrahydro-2H-pyran-4-yl)oxy)pyridin-3-yl)-2,3-dihydro-1H-inden-1-yl)oxy)phenyl)hex-4-ynoic acid;
(25) (S)-3-(4-(((R)-5-methoxy-4-(6-((tetrahydro-2H-pyran-4-yl)oxy)pyridin-3-yl)-2,3-dihydro-1H-inden-1-yl)oxy)phenyl)hex-4-ynoic acid;
(26) (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid; or
(27) 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid.

In an embodiment, the compound of Formula (I) is (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid of the following formula:

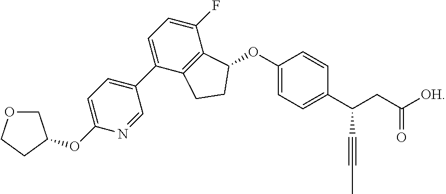 [SMILES: CC#C[C@@H](CC(=O)O)c1ccc(O[C@@H]2CCc3c(-c4ccc(O[C@@H]5CCOC5)nc4)ccc(F)c32)cc1]

In the above embodiments, the compound include an isomer or a pharmaceutically acceptable salt.

The isomer as used with regard to the compound of Formula (I) includes stereoisomers such as diastereomers, enantiomers, and atropisomers. The compound also includes mixtures of the stereoisomers such as racemic mixtures.

### Compositions and Treatments

The pharmaceutical composition may be formulated for oral, rectal, nasal, topical (including buccal and sublingual), transdermal, vaginal or parenteral (including intramuscular, subcutaneous and intravenous) administration in liquid or solid form or in a form suitable for administration by inhalation or insufflation. Oral administration is preferred. The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition and the dosage forms preferably comprises one or more pharmaceutical acceptable carriers. Preferred carriers must be “acceptable” in the sense of being compatible with the other ingredients of the formulation and not deleterious to the recipient thereof. Examples of pharmaceutically acceptable carriers are known to the one skilled in the art.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in molds.

In embodiments, methods and compositions for use in treating metabolic disorders disclosed herein are used to treat inadequate or insufficient glycemic control in a patient with a metabolic disorder. Inadequate or insufficient glycemic control may be considered to be a condition wherein patients exhibit HbA1c values above 5.7%, for example 5.7%-6.4% or above 6.0%, particularly above 6.5%, above 7.0%, above 7.5%, above 8%, above 8.5%, above 9%, above 9.5%, above 10%, above 10.5%, above 11%, or any value between 6.0% and 11.0%. For example, patients with inadequate or insufficient glycemic control may include patients having an HbA1c value from 5.7 to 6.4%, 6.5 to 7.0%, 7.0 to 7.5%, 7.5 to 10%, or from 7.5 to 11%. For example, inadequately controlled patients can refer to patients with poor glycemic control including, without being limited, patients having an HbA1c value≥9%.

In embodiments, methods and compositions for use in treating metabolic disorders disclosed herein lower HbA1c levels by an amount greater than 0.25%. In embodiments, methods and compositions for use in treating metabolic disorders disclosed herein lower HbA1c levels by an amount greater than 0.5%. In embodiments, methods and compositions for use in treating metabolic disorders disclosed herein lower HbA1c levels by an amount greater than 0.75%, or greater than 1%, or greater than 1.25%, or greater than 1.5%, or greater than 2%. In embodiments, methods and compositions for use in treating metabolic disorders disclosed herein may achieve a reduction of HbA1c levels in a range from about 0.25% to about 3%.

The pharmaceutical compositions may be administered as a fixed dose, at regular intervals, to achieve therapeutic efficacy. The pharmaceutical composition product's duration of action is typically reflected by its plasma half-life. Advantageously disclosed herein are methods of treating metabolic disorders such as diabetes or pre-diabetes by administration of the compound of Formula (I), its isomer, or a pharmaceutically acceptable salt thereof (collectively, “compound of Formula (I)”). For example, in embodiments, methods of treating a treating metabolic disorder are provided which include administering to a patient in need thereof a pharmaceutical composition including about 0.05 mg to about 100 mg, e.g., about 0.5 mg to about 50 mg, about 0.5 mg to about 30 mg, about 0.6 mg to about 30 mg, about 0.7 mg to about 30 mg, about 0.8 mg to about 30 mg, about 0.9 mg to about 30 mg, about 1 mg to about 30 mg, about 0.5 mg to about 25 mg, about 0.6 mg to about 25 mg, about 0.7 mg to about 25 mg, about 0.8 mg to about 25 mg, about 0.9 mg to about 25 mg, about 1 mg to about 25 mg, about 0.5 mg to about 20 mg, about 0.6 mg to about 20 mg, about 0.7 mg to about 20 mg, about 0.8 mg to about 20 mg, about 0.9 mg to about 20 mg, about 1 mg to about 20 mg, about 0.5 mg to about 10 mg, about 0.6 mg to about 10 mg, about 0.7 mg to about 10 mg, about 0.8 mg to about 10 mg, about 0.9 mg to about 10 mg, about 1 mg to about 10 mg, about 1.5 mg to about 10 mg, about 2 mg to about 10 mg, about 2.5 mg to about 10 mg, about 3 mg to about 10 mg, about 4 mg to about 10 mg, about 5 mg to about 10 mg, about 1.5 mg to about 30 mg, about 2 mg to about 30 mg, about 2.5 mg to about 30 mg, about 3 mg to about 30 mg, about 4 mg to about 30 mg, or about 5 mg to about 30 mg of compound of Formula (I), as a daily dose, wherein the composition provides improvement in glycemic control for more than 6 hours after administration to the patient. In embodiments, the compound of Formula (I) may be provided as a solvate such as a monohydrate or dehydrate. Thus, for example, 5.0, 10.0, or 15.0 mg of (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid would correspond to approximately 5.18, 10.36, and 15.54 mg of its monohydrate form.

In embodiments, methods of treating a metabolic disorder such as type 1 diabetes, type 2 diabetes or pre-diabetes include administering to a patient in need thereof a pharmaceutical composition including about 0.05 mg to about 50 mg of compound of Formula (I). In embodiments, methods of treating a metabolic disorder such as type 1 diabetes, type 2 diabetes or pre-diabetes include administering to a patient in need thereof a pharmaceutical composition including about 0.1 mg to about 30 mg of compound of Formula (I) or a pharmaceutically acceptable salt thereof. In embodiments, methods of treating a metabolic disorder such as type 1 diabetes, type 2 diabetes or pre-diabetes include administering to a patient in need thereof a pharmaceutical composition including about 0.5 mg to about 20 mg of compound of Formula (I). In embodiments, methods of treating a metabolic disorder such as type 1 diabetes, type 2 diabetes or pre-diabetes include administering to a patient in need thereof a pharmaceutical composition including about 0.5 mg to about 10 mg/day, about 1 mg to about 5 mg/day, about 5 mg to about 10 mg/day, about 10 mg to about 15 mg/day, about 15 mg to about 20 mg/day, about 20 mg to about 25 mg/day, about 25 mg to about 30 mg/day, about 1 mg/day, about 2 mg/day, about 3 mg/day, about 4 mg/day, about 5 mg/day, about 6 mg/day, about 7 mg/day, about 7.5 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 21 mg/day, about 22 mg/day, about 23 mg/day, about 24 mg/day, about 25 mg/day, about 26 mg/day, about 27 mg/day, about 28 mg/day, about 29 mg/day, or about 30 mg/day of compound of Formula (I) or a pharmaceutically acceptable salt thereof, isomer, or a mixture thereof.

In embodiments, the pharmaceutical compositions may include 0.1 mg to 30 mg, 0.1 mg to 25 mg, 0.1 mg to 20 mg, 0.1 mg to 15 mg, 0.1 mg to 10 mg, 0.5 mg to 25 mg, 0.5 mg to 20 mg, 0.5 to 15 mg, 0.5 mg to 10 mg, 1 mg to 25 mg, 1 mg to 20 mg, 1 mg to 15 mg, 1 mg to 10 mg, 1.5 mg to 25 mg, 1.5 mg to 20 mg, 1.5 mg to 15 mg, 1.5 mg to 10 mg, 2 mg to 25 mg, 2 mg to 20 mg, 2 mg to 15 mg, 2 mg to 10 mg, 2.5 mg to 25 mg, 2.5 mg to 20 mg, 2.5 mg to 15 mg, 2.5 mg to 10 mg, 3 mg to 25 mg, 3 mg to 20 mg, 3 mg to 15 mg, 3 mg to 10 mg, 4 mg to 25 mg, 4 mg to 20 mg, 4 mg to 15 mg, 4 mg to 10 mg, 5 mg to 25 mg, 5 mg to 20 mg, 5 mg to 15 mg, or 5 mg to 10 mg of compound of Formula (I), as a daily dose. Herein, the compound of Formula (I) includes an isomer, a pharmaceutically acceptable salt, or a mixture thereof.

In embodiments, the pharmaceutical compositions can include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg of compound of Formula (I), as a daily dose. Herein, the compound of Formula (I) includes an isomer, a pharmaceutically acceptable salt, or a mixture thereof.

In embodiments, the pharmaceutical compositions can include 0.1 mg, 0.25 mg, 0.5 mg, 1 mg, 1.5 mg, 2.0 mg, 2.5 mg, 3 mg, 4 mg, 5 mg, 6 mg, 7 mg, 7.5 mg, 8 mg, 9 mg, 10 mg, 11 mg, 12 mg, 12.5 mg, 13 mg, 14 mg, 15 mg, 16 m, 17 mg, 17.5 mg, 18 mg, 19 mg, or 20 mg of compound of Formula (I) or amounts that are multiples of such doses. In embodiments, the pharmaceutical compositions include 0.5 mg, 1 mg, 2 mg, 2.5 mg, 3 mg, 4 mg, 5 mg, 6 mg, 7 mg, 7.5 mg, 8 mg, 9 mg, 10 mg, 12 mg, 15 mg, or 20 mg of compound of Formula (I), as a daily dose. Herein, the compound of Formula (I) includes an isomer, a pharmaceutically acceptable salt, or a mixture thereof.

Pharmaceutical compositions herein may be provided with conventional release profiles or modified release profiles. Conventional (or unmodified) release oral dosage forms such as tablets or capsules typically release medications into the stomach or intestines as the tablet or capsule shell dissolves. The pattern of drug release from modified release dosage forms is deliberately changed from that of a conventional dosage form to achieve a desired therapeutic objective and/or better patient compliance. Types of modified release drug products include orally disintegrating dosage forms which provide immediate release, extended release dosage forms, delayed release dosage forms (e.g., enteric coated), and pulsatile release dosage forms. In embodiments, pharmaceutical compositions with different drug release profiles may be combined to create a two phase or three-phase release profile. For example, pharmaceutical compositions may be provided with an immediate release and an extended release profile. In embodiments, pharmaceutical compositions may be provided with an extended release and delayed release profile. Such composition may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions.

Orally disintegrating dosage forms disintegrate quickly when in contact with saliva. They can be in a tablet form or rapidly dissolving films that are thin oral strips that release medication after administration to the oral cavity.

In embodiments, pharmaceutical compositions having modified release profiles provide pharmacokinetic properties which result in both rapid onset and sustained duration of action. Such pharmaceutical compositions include an immediate release aspect and an extended release aspect. Immediate release aspects are discussed above in connection with orally disintegrating dosage forms. Extended release dosage forms have extended release profiles and are those that allow a reduction in dosing frequency as compared to that presented by a conventional dosage form, e.g., a solution or unmodified release dosage form. Extended release dosage forms provide a sustained duration of action of a drug. In embodiments, modified release dosage forms herein are extended release dosage forms that do not have an orally disintegrating dosage form aspect. In embodiments, modified release dosage forms may provide immediate release of a loading dose and then an extended release dosage forms aspect that provides prolonged delivery to maintain drug levels in the blood within a desired therapeutic range for a desirable period of time in excess of the activity resulting from a single dose of the drug. In embodiments, the orally disintegrating dosage form aspect releases the drug immediately and the extended release dosage form aspect thereafter provides continuous release of drug for sustained action.

In embodiments, modified release pharmaceutical compositions include pulsatile release dosage formulations. Pulsatile drug release involves rapid release of defined or discrete amounts of a drug (or drugs) after a lag time following an initial release of drug. In embodiments, pulsatile release dosage forms can provide a single pulse. In embodiments, pulsatile release dosage forms can provide multiple pulses over time. Various pulsatile release dosage forms are known to those with skill in the art.

In embodiments, the modified release pharmaceutical compositions may include 0.1 mg to 75 mg, 0.1 mg to 70 mg, 0.1 mg to 65 mg, 0.1 mg to 55 mg, 0.1 mg to 50 mg, 0.1 mg to 45 mg, 0.1 mg to 40 mg, 0.1 mg to 35 mg, 0.1 mg to 30 mg, 0.1 mg to 25 mg, 0.1 mg to 20 mg, 0.1 mg to 15 mg, 0.1 mg to 10 mg, 0.5 mg to 75 mg, 0.5 mg to 70 mg, 0.5 mg to 65 mg, 0.5 mg to 55 mg, 0.5 mg to 50 mg, 0.5 mg to 45 mg, 0.5 mg to 40 mg, 0.5 mg to 35 mg, 0.5 mg to 30 mg, 0.5 mg to 25 mg, 0.5 mg to 20 mg, 0.5 to 15 mg, 0.5 to 10 mg, 1 mg to 75 mg, 1 mg to 70 mg, 1 mg to 65 mg, 1 mg to 55 mg, 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1.5 mg to 75 mg, 1.5 mg to 70 mg, 1.5 mg to 65 mg, 1.5 mg to 55 mg, 1.5 mg to 50 mg, 1.5 mg to 45 mg, 1.5 mg to 40 mg, 1.5 mg to 35 mg, 1.5 mg to 30 mg, 1.5 mg to 25 mg, 1.5 mg to 20 mg, 1.5 mg to 15 mg, 1.5 mg to 10 mg, 2 mg to 75 mg, 2 mg to 70 mg, 2 mg to 65 mg, 2 mg to 55 mg, 2 mg to 50 mg, 2 mg to 45 mg, 2 mg to 40 mg, 2 mg to 35 mg, 2 mg to 30 mg, 2 mg to 25 mg, 2 mg to 20 mg, 2 mg to 15 mg, 2 mg to 10 mg, 2.5 mg to 75 mg, 2.5 mg to 70 mg, 2.5 mg to 65 mg, 2.5 mg to 55 mg, 2.5 mg to 50 mg, 2.5 mg to 45 mg, 2.5 mg to 40 mg, 2.5 mg to 35 mg, 2.5 mg to 30 mg, 2.5 mg to 25 mg, 2.5 mg to 20 mg, 2.5 mg to 15 mg, 2.5 mg to 10 mg, 3 mg to 75 mg, 3 mg to 70 mg, 3 mg to 65 mg, 3 mg to 55 mg, 3 mg to 50 mg, 3 mg to 45 mg, 3 mg to 40 mg, 3 mg to 35 mg, 3 mg to 30 mg, 3 mg to 25 mg, 3 mg to 20 mg, 3 mg to 15 mg, 3 mg to 10 mg, 3.5 mg to 75 mg, 3.5 mg to 70 mg, 3.5 mg to 65 mg, 3.5 mg to 55 mg, 3.5 mg to 50 mg, 3.5 mg to 45 mg, 3.5 mg to 40 mg, 3.5 mg to 35 mg, 3.5 mg to 30 mg, 3.5 mg to 25 mg, 3.5 mg to 20 mg, 3.5 mg to 15 mg, 3.5 mg to 10 mg, 4 mg to 75 mg, 4 mg to 70 mg, 4 mg to 65 mg, 4 mg to 55 mg, 4 mg to 50 mg, 4 mg to 45 mg, 4 mg to 40 mg, 4 mg to 35 mg, 4 mg to 30 mg, 4 mg to 25 mg, 4 mg to 20 mg, 4 mg to 15 mg, 4 mg to 10 mg, 4.5 mg to 75 mg, 4.5 mg to 70 mg, 4.5 mg to 65 mg, 4.5 mg to 55 mg, 4.5 mg to 50 mg, 4.5 mg to 45 mg, 4.5 mg to 40 mg, 4.5 mg to 35 mg, 4.5 mg to 30 mg, 4.5 mg to 25 mg, 4.5 mg to 20 mg, 4.5 mg to 15 mg, 4.5 mg to 10 mg, 5 mg to 75 mg, 5 mg to 70 mg, 5 mg to 65 mg, 5 mg to 55 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, or 5 mg to 10 mg, of the compound of Formula (I), as a daily dose. Herein, the compound of Formula (I) includes an isomer, a pharmaceutically acceptable salt, or a mixture thereof.

In embodiments, pharmaceutical compositions may include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg of the compound of Formula (I), as a daily dose. Herein, the compound of Formula (I) includes an isomer, a pharmaceutically acceptable salt, or a mixture thereof.

In embodiments, pharmaceutical compositions may include 0.1 mg, 0.25 mg, 0.5 mg, 1 mg, 2.5 mg, 3 mg, 4 mg, 5 mg, 7 mg, 7.5 mg, 8 mg, 9 mg, 10 mg, 11 mg, 12 mg, 12.5 mg, 13 mg, 14 mg, 15 mg, 16 mg, 17 mg, 17.5 mg, 18 mg, 19 mg, or 20 mg of the compound of Formula (I) or amounts that are multiples of such doses. In embodiments, pharmaceutical compositions may include 2.5 mg, 5 mg, 7.5 mg, 10 mg, 12.5 mg, 15 mg, or 20 mg of the compound of Formula (I) as a daily dose. Herein, the compound of Formula (I) includes an isomer, a pharmaceutically acceptable salt, or a mixture thereof.

In embodiments, orally disintegrating dosage forms may include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 7 mg, 7.5 mg, 8 mg, 9 mg, 10 mg, 11 mg, 12 mg, 12.5 mg, 13 mg, 14 mg, 15 mg, 16 mg, 17 mg, 17.5 mg, 18 mg, 19 mg, or 20 mg of the compound of Formula (I), or amounts that are multiples of such doses, as a daily dose. Herein, the compound of Formula (I) includes an isomer, a pharmaceutically acceptable salt, or a mixture thereof.

In embodiments, extended release dosage forms may include from about 1 mg to about 100 mg of the compound of Formula (I). In embodiments, extended release dosage forms may include 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of the compound of Formula (I). Herein, the compound of Formula (I) includes an isomer, a pharmaceutically acceptable salt, or a mixture thereof.

In embodiments, delayed release dosage forms may include from about 0.05 mg to about 100 mg of the compound of Formula (I). In embodiments, delayed release dosage forms include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of the compound of Formula (I), as a daily dose. Herein, the compound of Formula (I) includes an isomer, a pharmaceutically acceptable salt, or a mixture thereof.

In embodiments, pulsatile release dosage forms may include one or more pulse providing domains having from about 0.05 mg to about 100 mg of the compound of Formula (I). In embodiments, pulsatile release dosage form may include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of the compound of Formula (I), as a daily dose. Herein, the compound of Formula (I) includes an isomer, a pharmaceutically acceptable salt, or a mixture thereof.

In embodiments, the pharmaceutical compositions described herein are administered once, twice, or three times daily, or every other day. In embodiments, a pharmaceutical composition described herein may be administered to the patient in the evening. In embodiments, a pharmaceutical composition may be administered to the patient in the morning. In embodiments, a pharmaceutical composition may be administered to the patient once in the evening and once in the morning. In embodiments, the total amount of compound of Formula (I) administered to a subject in a 24-hour period is 0.5 mg to 30 mg. In embodiments, the total amount of compound of Formula (I) administered to a subject in a 24-hour period is 0.05 mg to 30 mg, e.g., 0.5 mg to 20 mg or 0.5 mg to 10 mg. In embodiments, the total amount of compound of Formula (I) or a pharmaceutically acceptable salt thereof administered to a subject in a 24-hour period is 0.1 mg, 0.25 mg, 0.5 mg, 1 mg, 2 mg, 5 mg, 6 mg, 7 mg, 7.5 mg, 8 mg, 9 mg, 10 mg, 12.5 mg, 15 mg, 17.5 mg, or 20 mg. In embodiments, the total amount of compound of Formula (I) administered to a subject in a 24-hour period may be 20 mg. In embodiments, the subject may be started at a low dose and the dosage is escalated. In this manner, it can be determined if the drug is well tolerated in the subject. In embodiments, the effect of compound of Formula (I), either alone or in combination with a hypoglycemic agent, is adjusted according to the patient's response. Dosages can be lower for children than for adults.

In embodiments, methods of treating a metabolic disorder such as type 1 diabetes, type 2 diabetes or pre-diabetes comprise administering to a patient in need thereof a pharmaceutical composition including compound of Formula (I) wherein the composition provides improvement in at least one symptom of the metabolic disorder.

In embodiments, methods of treating a metabolic disorder such as type 1 diabetes, type 2 diabetes or pre-diabetes includes administering to a patient in need thereof a pharmaceutical composition including compound of Formula (I) wherein the composition provides improvement of at least one symptom for more than 4 hours after administration of the pharmaceutical composition to the patient. In embodiments, provided herein is improvement of at least one symptom for more than 6 hours after administration of the pharmaceutical composition to the patient. In embodiments, provided herein is improvement of at least one symptom for more than, e.g., 8 hours, 10 hours, 12 hours, 15 hours, 18 hours, 20 hours, or 24 hours after administration of the pharmaceutical composition to the patient. In embodiments, provided herein is improvement in at least one symptom for at least, e.g., 8 hours, 10 hours, 12 hours, 15 hours, 18 hours, 20 hours, or 24 hours after administration of the pharmaceutical composition to the patient. In embodiments, provided herein is improvement in at least one symptom for 12 hours after administration of the pharmaceutical composition to the patient.

In embodiments, methods of treating a metabolic disorder such as type 1 diabetes, type 2 diabetes or pre-diabetes comprise administration of compound of Formula (I) in combination with one or more other active agents. The combination therapies can include administration of the active agents together in the same admixture, or in separate admixtures. In embodiments, the pharmaceutical composition includes two, three, or more active agents. In embodiments, the combinations result in a more than additive effect on the treatment of the disease or disorder. For example, the combination of the compound of Formula (I) and one or more hypoglycemic agents provides a therapeutic benefit greater than the additive effect of administering the same dosage of each of the compound of Formula (I) and the hypoglycemic agents alone. Thus, treatment is provided of a metabolic disorder with a combination of agents that combined, may provide a synergistic effect that enhances efficacy.

In embodiments, administration of compound of Formula (I), alone or optionally in combination with one or more hypoglycemic agents such as a biguanide, a dipeptidyl peptidase-4 (DPP-4) inhibitor, a sulphonylurea, a thiazolidinedione, a meglitinide glinide), an alpha-glucosidase blocker, a glucagon-like peptide-1 (GLP-1) receptor agonist, insulin or an insulin analog to a patient in need thereof is provided. In embodiments, a pharmaceutical composition of compound of Formula (I), alone or optionally in combination with one or more hypoglycemic agents such as a biguanide, a dipeptidyl peptidase-4 (DPP-4) inhibitor, a sulphonylurea, a thiazolidinedione, a meglitinide (glinide), an alpha-glucosidase blocker, a GLP-1 receptor agonist, insulin or an insulin analog is provided.

In embodiments, methods of treating a metabolic disorder such as type 1 diabetes, type 2 diabetes or pre-diabetes herein include administering to the patient in need thereof the compound of Formula (I) in combination with about 50 mg to about 3000 mg of metformin or a pharmaceutically acceptable salt thereof. In embodiments, about 50 mg to about 3000 mg of metformin or a pharmaceutically acceptable salt thereof may be administered in 24 hours. In embodiments, the metformin or a pharmaceutically acceptable salt thereof may be administered in divided doses over 24 hours. In embodiments, metformin may be administered once a day, e.g., with an evening meal. In embodiments, metformin can be given in doses varying from about 500 mg to 2000 mg up to 2500 mg or 3000 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or 500 mg to 1000 mg once or twice a day or about 500 mg to 2000 mg once a day.

In embodiments, methods of treating a metabolic disorder such as type 1 diabetes, type 2 diabetes or pre-diabetes include administering to a patient in need thereof of the compound of Formula (I), in combination with insulin or an insulin analog. The insulin can be commercially available fast acting insulin analogs, e.g., lispro or glulisine, short acting (regular) insulin, intermediate acting (NPH) insulin, long acting insulin, e.g., glargine or detemir, ultra-long acting, e.g., degludec, or combination insulin products. Insulin or insulin analogs may be administered parenterally, e.g., subcutaneously. Short acting or regular human insulin may be available in two concentrations: 100 units of insulin per mL (U-100) and 500 units of insulin per mL (U-500). Insulin may be administered as a fixed dose or as a flexible dose therapy. Factors which may affect insulin dosage include carbohydrate intake, physical activity, illness, body mass and insulin resistance. Typically, insulin doses are individualized based on metabolic needs and frequent monitoring of blood glucose. In general, total daily insulin requirements can be between 0.5 to 1 unit/kg/day.

### EXAMPLES

The Examples provided herein are included solely for augmenting the disclosure herein and should not be considered to be limiting in any respect.

### Reference Example 1: (S)-3-(4-(((R)-7-Fluoro-4-(6-(((R)-tetrahydrofuran-3-yl)oxy)pyridin-3-yl)-2,3-dihydro-1H-inden-1-yl)oxy)phenyl)hex-4-ynoic acid

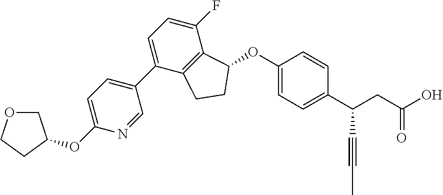 [SMILES: CC#C[C@@H](CC(=O)O)c1ccc(O[C@@H]2CCc3c(-c4ccc(O[C@@H]5CCOC5)nc4)ccc(F)c32)cc1]

2.0 M aqueous lithium hydroxide solution (5.0 eq.) was added to a solution of (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid methyl ester (1.0 eq.) in tetrahydrofuran (1.0 M) and methanol (4.0 M) at 4° C. The mixture was stirred at room temperature for 18 h. The mixture was neutralized with saturated aqueous ammonium chloride solution and diluted with ethyl acetate.

The organic layer was washed with brine, dried over magnesium sulfate, filtered, and concentrated. The resultant residue was purified by flash column chromatography on silica gel to afford (S)-3-(4-(((R)-7-Fluoro-4-(6-(((R)-tetrahydrofuran-3-yl)oxy)pyridin-3-yl)-2,3-dihydro-1H-inden-1-yl)oxy)phenyl)hex-4-ynoic acid. MS ESI (positive) m/z: 502.24 (M+H).

¹H NMR (400 MHz, CDCl₃) δ 8.15 (d, J=2.4 Hz, 1H), 7.64 (dd, J=8.6, 2.6 Hz, 1H), 7.38-7.26 (m, 3H), 7.03 (t, J=8.6 Hz, 1H), 6.98-6.93 (m, 2H), 6.81 (dd, J=8.4, 0.4 Hz, 1H), 5.95-5.91 (m, 1H), 5.61-5.58 (m, 1H), 4.11-3.89 (m, 5H), 3.29-3.19 (m, 1H), 2.91-2.71 (m, 3H), 2.42-2.15 (m, 4H), 1.84 (d, J=2.4 Hz, 3H).

### Reference Example 2

Coated tablets containing 7.5 mg of active substance are produced.

1 tablet core contains:

| | | |
|---|---|---|
| active substance | 7.5 | mg |
| calcium phosphate | 9.3 | mg |
| corn starch | 3.6 | mg |
| polyvinylpyrrolidone | 1.0 | mg |
| methylcellulose | 1.5 | mg |
| magnesium stearate | 0.1 | mg |
| | 23 | mg |

The active substance is mixed with calcium phosphate, corn starch, polyvinylpyrrolidone, methylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape. The tablet cores thus produced are coated with a film consisting essentially of methylcellulose. The finished film-coated tablets are polished with beeswax.

### Reference Example 3

Tablets containing 10 mg of active substance are produced.

1 tablet contains:

| | | |
|---|---|---|
| active substance | 10.0 | mg |
| lactose | 8.0 | mg |
| maize starch | 3.4 | mg |
| polyvinylpyrrolidone | 0.4 | mg |
| magnesium stearate | 0.2 | mg |
| | 22.0 | mg |

The active substance, lactose and starch are mixed together and uniformly moistened with an aqueous solution of the polyvinylpyrrolidone. After the moist composition has been screened (2.0 mm mesh size) and dried in a rack-type drier at 50° C. It is screened again (1.5 mm mesh size) and the lubricant is added. The finished mixture is compressed to form tablets.

### Example 1: Pharmacokinetic and Pharmarcodynamic Data (PK/PD) Study

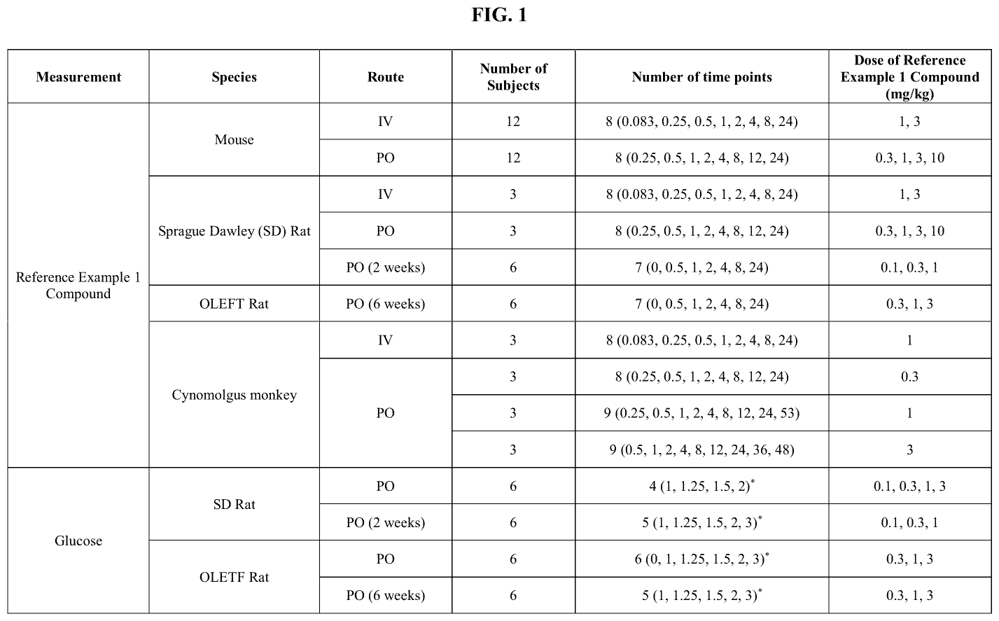

Pharmacokinetic and pharmarcodynamic data (PK/PD) were collected using the animal species shown in FIG. 1. As the test compound of Formula (I), the compound prepared in Reference Example 1 was employed. Reference Example 1 compound or glucose was provided to the animals through IV or PO routes at the frequency and doses shown in FIG. 1. Data were collected after single dosing and repeated dosings.

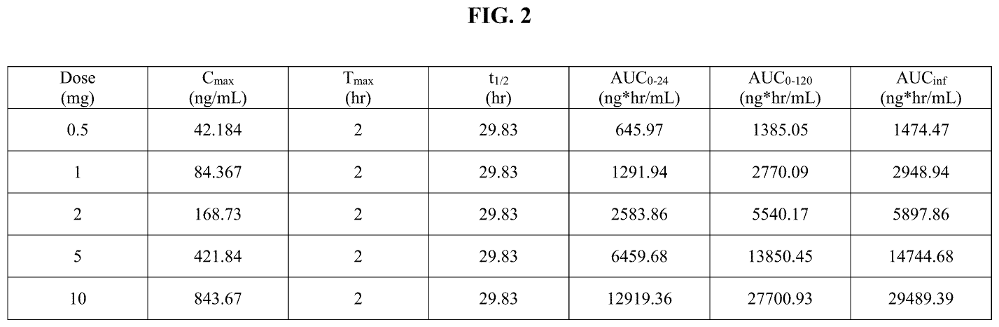

Employing the PK/PD data obtained from mouse, rats (SD rats and OLEFT rats) and cynomolgus monkey, simulated human PK parameters and PD parameters were calculated for a male human of 70 kg body weight. The obtained simulated human PK parameters of Reference Example 1 compound are shown in FIG. 2.

To predict glucose-lowering effect of Reference Example 1 compound compared to vehicle, PD simulation was performed using human PK parameters obtained from interspecies allometry scaling as shown in FIG. 2. The inventors simulated glucose concentration-time profiles of OGTT (oral glucose tolerance testing) after repeated administration of Reference Example 1 compound for 2 weeks with dose range of 0.5-10 mg. 3 g of glucose was administered at 1 hour after the drug (Reference Example 1 compound administration). The glucose baseline was set as 150 mg/dL assuming disease condition.

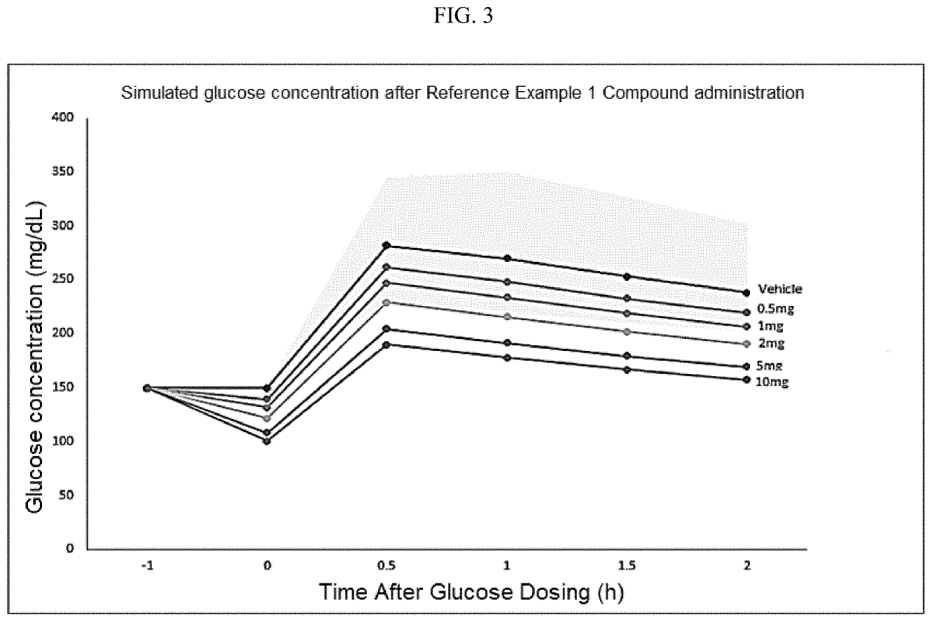

Simulated glucose concentration-time profiles after drug and glucose administration are obtained (FIG. 3). Based on the simulation, glucose AUC reduction rates compared to vehicle by dose level are summarized in Table 1.

TABLE 1

| | Glucose AUC |
|---|---|
| Dose (mg) | reduction (%) |
| 0.5 | 7.7 |
| 1 | 13.0 |
| 2 | 19.7 |
| 5 | 28.6 |
| 10 | 33.6 |

### Example 2: Oral Glucose Tolerance Test

An oral glucose tolerance test is performed in overnight fasted male Sprague Dawley (SD) rats (Crl:CD(SD)) with a body weight of about 200 g. A pre-dose blood sample is obtained by tail bleed. Blood glucose is measured with a glucometer, and the animals are randomized for blood glucose (n=5/group). Subsequently, the groups receive a single oral administration of either vehicle alone (0.5% methylcellulose in distilled water) or vehicle containing either the compound of Formula (I) or the second or third hypoglycemic agent or the combination of the compound of Formula (I) plus the second plus, optionally, the third antidiabetic agent. As the compound of Formula (I), Reference Example 1 compound could be used.

Alternatively the groups receive a single oral administration of either vehicle alone or vehicle containing either the compound of Formula (I) or the second hypoglycemic agent plus the third hypoglycemic agent or the combination of the compound of Formula (I) plus the second hypoglycemic agent plus the third hypoglycemic agent. The animals receive an oral glucose load (2 g/kg) 30 min after compound administration. Blood glucose is measured in tail blood 15, 30, 60, and 120 min after the glucose challenge. Glucose excursion is quantified by calculating the reactive glucose AUC. The data are presented as mean±S.E.M. Statistical comparisons are conducted by Graph Pad Prism program, One or Two-way ANOVA test.

### Example 3: Treatment of Pre-Diabetes

The efficacy of a pharmaceutical composition or combination according to the invention in the treatment of pre-diabetes characterized by pathological fasting glucose and/or impaired glucose tolerance can be tested using clinical studies. Five groups of patients (each group including 10 patients) are administered daily with 0.5-20 mg of the compound of Formula (I) (e.g., Reference Example 1 compound), respectively. In studies over a shorter period (e.g. 2-4 weeks) the success of the treatment is examined by determining the fasting glucose values and/or the glucose values after a meal or after a loading test (oral glucose tolerance test or food tolerance test after a defined meal) after the end of the period of therapy for the study and comparing them with the values before the start of the study and/or with those of a placebo group. In addition, the fructosamine value can be determined before and after therapy and compared with the initial value and/or the placebo value. A significant drop in the fasting or non-fasting glucose levels demonstrates the efficacy of the treatment. In studies over a longer period (12 weeks or more) the success of the treatment is tested by determining the HbA1c value, by comparison with the initial value and/or with the value of the placebo group. A significant change in the HbA1c value compared with the initial value and/or the placebo value demonstrates the efficacy of the compound of Formula (I) or combinations according to the embodiments for treating pre-diabetes.

### Example 4: Prevention of Manifest Type 2 Diabetes

The efficacy of a treatment can be investigated in a comparative clinical study in which pre-diabetes patients are treated over a lengthy period (e.g. 1-5 years) with either a pharmaceutical composition or combination according to this invention or with placebo or with a non-drug therapy or other medicaments. During and at the end of the therapy, by determining the fasting glucose and/or a loading test (e.g. OGTT), a check is made to determine how many patients exhibit manifest type 2 diabetes, i.e. a fasting glucose level of >125 mg/dl and/or a 2 h value according to OGTT of >199 mg/dl. A significant reduction in the number of patients who exhibit manifest type 2 diabetes when treated with the compound of Formula (I) or combination according to the present invention as compared to one of the other forms of treatment, demonstrates the efficacy in preventing a transition from pre-diabetes to manifest diabetes.

### Example 5: Treatment of Type 2 Diabetes

The efficacy and safety of once-daily compound of Formula (I) (e.g., Reference Example 1 compound) monotherapy administered orally at 0.5-20 mg doses vs. placebo over a 6-month period are investigated for treatment-naive patients with type 2 diabetes inadequately controlled with diet and exercise. This may be a multicenter, randomized, four-arm, parallel-group, double-blind, placebo-controlled trial. Patients between 18 and 77 years old with type 2 diabetes inadequately controlled with diet and exercise (HbA1c greater than or equal to 7.0% at screening visit) will be eligible. Patients with a screening HbA1c greater than 7.0% and less than 10.0% may encompass the main treatment cohort (MTC). Patients with an HbA1c greater than 10.0% and less than 12.0% who otherwise meet all other inclusion and exclusion criteria are eligible for direct entry into the open-label cohort (OLC). For eligibility, all patients are required to be treatment naive (defined as never receiving medical treatment for diabetes [insulin and/or oral hypoglycemic medication] for greater than 6 months after original diagnosis, and no oral hypoglycemic medication for more than 3 consecutive days or 7 nonconsecutive days during the 8 weeks prior to screening), have a fasting C-peptide greater than or equal to 1 ng/mL (greater than or equal to 0.33 nmol/L), and a body mass index (BMI) less than or equal to 40 kg/m².

Following screening, MTC patients may enter a single-blind 2-week dietary and exercise placebo lead-in period. Patients who meet entrance criteria and demonstrate adequate compliance (80 to 120% of prescribed drug consumption) with study medication (placebo) during the lead-in period will qualify for enrollment. Patients are randomized to oral Reference Example 1 compound 0.5-20 mg, or placebo and are followed for 24 weeks on double-blind study medication. Patients enrolled in the OLC are entered directly into a 24-week treatment period where they receive oral, open-label Reference Example 1 compound at a dose of 20 mg once daily.

The primary endpoint may be change in HbA1c from baseline to week 24. Secondary endpoints may include change from baseline to week 24 in: (1) fasting plasma glucose (FPG); (2) proportion of patients achieving an HbA1 less than 7.0%; and (3) change from baseline in area under the curve (AUC) from 0 to 180 min for postprandial glucose (PPG) in response to a 75-g oral glucose tolerance test (OGTT). Other prespecified efficacy outcome measures may be PPG change from baseline at 120 min in response to an OGTT and changes from baseline to week 24 in levels of both fasting and postprandial insulin, C-peptide, and glucagon levels. B-Cell function are measured by homeostasis model assessment (HOMA)-2 and insulin resistance.

Efficacy analyses are performed on the randomized patients dataset, which may consist of randomized patients who receive at least one dose of study medication and who have a baseline and at least one post-baseline measurement. Each compound (I) group are compared with placebo for changes from baseline to week 24 in continuous variables utilizing an analysis of covariance (ANCOVA) model with treatment group as an effect and baseline value as a covariate. The percentage of patients achieving target HbA1c at week 24 are compared between each Reference Example 1 compound treatment group vs. placebo, using a two-sided Fisher exact test. Demographic and other baseline characteristics are summarized using descriptive statistics. Estimated average glucose (eAG) values are calculated post hoc based on HbA1c values using the following linear regression: eAG_{mg/dL}=28.7×HbA1c−46.7. Within the framework of the ANCOVA model, point estimates and 95% confidence intervals (CIs) for the absolute and adjusted mean change within each treatment group, as well as for the differences in mean changes between each of the compound (I) treatment groups (0.5-20 mg) and the placebo group, are calculated. For the primary endpoint, each comparison between a Reference Example 1 compound treatment group and the placebo group are performed at the alpha=0.019 level from Dunnett's adjustment so that the overall type I error rate are controlled at the 0.05 significance level. Sequential testing methodology are utilized for secondary efficacy endpoints to adjust for multiplicity and preserve the overall type I error rate within each treatment group at the 0.05 level.

### Example 6: Treatment of Insulin Resistance

In clinical studies running for different lengths of time (e.g. 2 weeks to 12 months) the success of the treatment is checked using a hyperinsulinemic euglycemic glucose clamp study. A significant rise in the glucose infusion rate at the end of the study, compared with the initial value or compared with a placebo group, or a group given a different therapy, proves the efficacy of compound of Formula (I), pharmaceutical composition or combination according to the embodiments in the treatment of insulin resistance.

### Example 7: Treatment of Hyperglycemia

0.5-20 mg clinical studies running for different lengths of time (e.g. 1 day to 24 months) the success of the treatment in patients with Hyperglycemia is checked by determining the fasting glucose or non-fasting glucose (e.g. after a meal or a loading test with OGTT or a defined meal). A significant fall in these glucose values during or at the end of the study, compared with the initial value or compared with a placebo group, or a group given a different therapy, proves the efficacy of compound of Formula (I), pharmaceutical composition or combination according to the present invention according to the invention in the treatment of hyperglycemia.

### Example 8: Prevention of Micro- or Macrovascular Complications

The treatment of type 2 diabetes or pre-diabetes patients with compound of Formula (I), pharmaceutical composition or combination according to embodiments prevents or reduces or reduces the risk of developing microvascular complications (e.g. diabetic neuropathy, diabetic retinopathy, diabetic nephropathy, diabetic foot, diabetic ulcer) or macrovascular complications (e.g. myocardial infarct, acute coronary syndrome, unstable angina pectoris, stable angina pectoris, stroke, peripheral arterial occlusive disease, cardiomyopathy, heart failure, heart rhythm disorders, vascular restenosis). Type 2 diabetes or patients with pre-diabetes are treated long-term, e.g. for 1-6 years, with a pharmaceutical composition or combination of embodiments and compared with patients who have been treated with other antidiabetic medicaments or with placebo.

Evidence of the therapeutic success compared with patients who have been treated with other antidiabetic medicaments or with placebo can be found in the smaller number of single or multiple complications. In the case of macrovascular events, diabetic foot and/or diabetic ulcer, the numbers are counted by anamnesis and various test methods. In the case of diabetic retinopathy the success of the treatment is determined by computer-controlled illumination and evaluation of the background to the eye or other ophthalmic methods. In the case of diabetic neuropathy, in addition to anamnesis and clinical examination, the nerve conduction rate can be measured using a calibrated tuning fork, for example. With regard to diabetic nephropathy the following parameters may be investigated before the start, during and at the end of the study: secretion of albumin, creatinine clearance, serum creatinine values, time taken for the serum creatinine values to double, time taken until dialysis becomes necessary.

### Example 9: Treatment of Metabolic Syndrome

A significant reduction in systolic and/or diastolic blood pressure, a lowering of the plasma triglycerides, a reduction in total or LDL cholesterol, an increase in HDL cholesterol or a reduction in weight, either compared with the starting value at the beginning of the study or in comparison with a group of patients treated with placebo or a different therapy proves the efficacy of compound of Formula (I) or a combination with other hypoglycemic agent in the treatment of metabolic syndrome.

### Example 10: Clinical Study (Single Ascending Dose (SAD) Study in Healthy Human)

To determine safety and pK data in human subjects, single ascending dose (SAD) study was conducted. SAD study included 5 cohorts of 8 healthy subjects who were randomized and received a single oral dose of Reference Example 1 compound or placebo (3:1) at 0.5 mg, 1 mg, 2 mg, 5 mg, or 10 mg. Each cohort was initiated after review of the safety and PK data of the previous cohort. 5 mg cohort was divided into two groups, wherein one group was administered with Reference Example 1 compound in fasted state and the other group was administered with Reference Example 1 compound together with food.

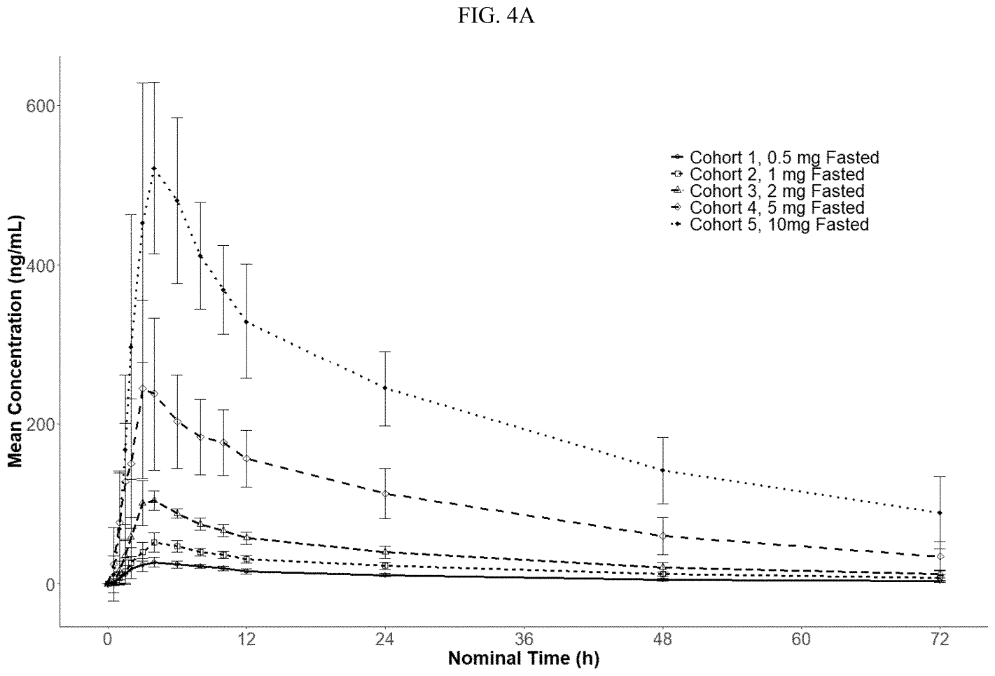

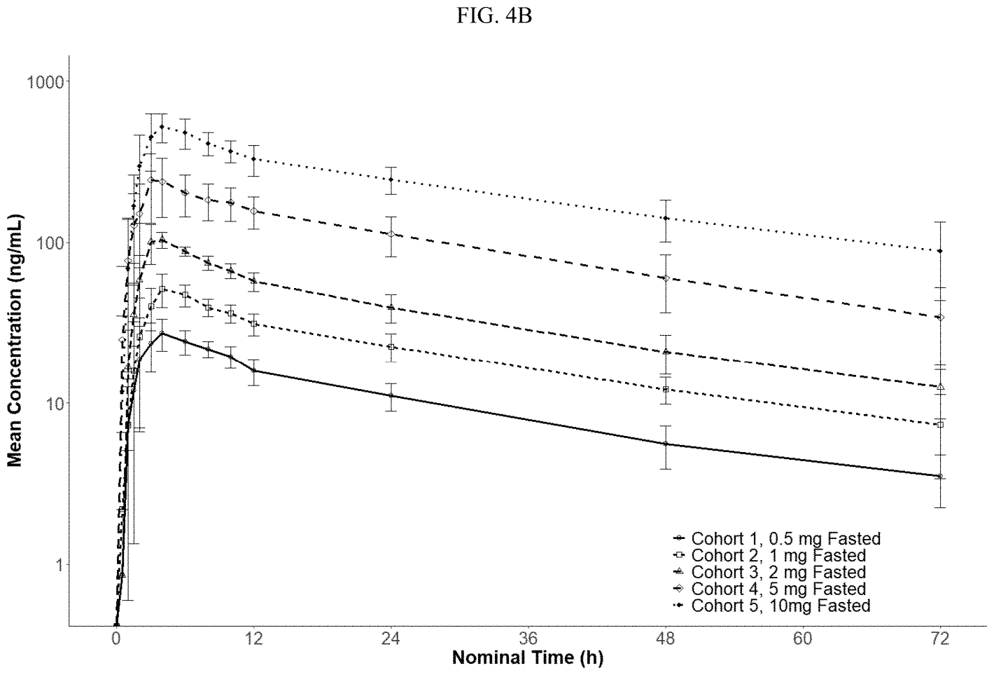

Mean plasma concentration of Reference Example 1 compound following single dose of 0.5, 1, 2, 5, and 10 mg are shown in FIG. 4A (linear scale) and FIG. 4B (semi-logarithmic scale). Cₘₐₓ, AUCₗₐₛₜ, and AUC_{inf} for each cohort are shown in Table 2.

TABLE 2

| | | | | | 5 mg | 5 mg | |
|---|---|---|---|---|---|---|---|
| Parameter | Statistic | 0.5 mg | 1 mg | 2 mg | Fasted | Fed | 10 mg |
| Cₘₐₓ | N | 6 | 6 | 6 | 6 | 6 | 6 |
| (ng/mL) | Geometric | 28.37 | 55.83 | 108.59 | 242.69 | 250.44 | 544.84 |
| | Mean | | | | | | |
| | Geometric | 20.2 | 15.4 | 15.2 | 42.8 | 21.3 | 24.5 |
| | CV % | | | | | | |
| AUCₗₐₛₜ | N | 6 | 6 | 6 | 6 | 6 | 6 |
| (h*ng/mL) | Geometric | 680.33 | 1,365.52 | 2,480.35 | 6,519.42 | 6,928.72 | 14,792.63 |
| | Mean | | | | | | |
| | Geometric | 21.1 | 13.4 | 16.7 | 35.3 | 32.6 | 25.9 |
| | CV % | | | | | | |
| AUC_{inf} | N | 6 | 6 | 6 | 6 | 6 | 6 |
| (h*ng/mL) | Geometric | 811.26 | 1,662.47 | 2,975.58 | 7,740.99 | 8,272.66 | 18,575.12 |
| | Mean | | | | | | |
| | Geometric | 26.4 | 23.9 | 21.8 | 43.4 | 33.6 | 31.7 |
| | CV % | | | | | | |

In the first-in-human study, single doses from 0.5 mg to 10 mg were well tolerated, when administered while fasting. As expected from preclinical PK modelling, none of the single doses exceeded the PK stopping criteria for Cₘₐₓ and AUCₗₐₛₜ.

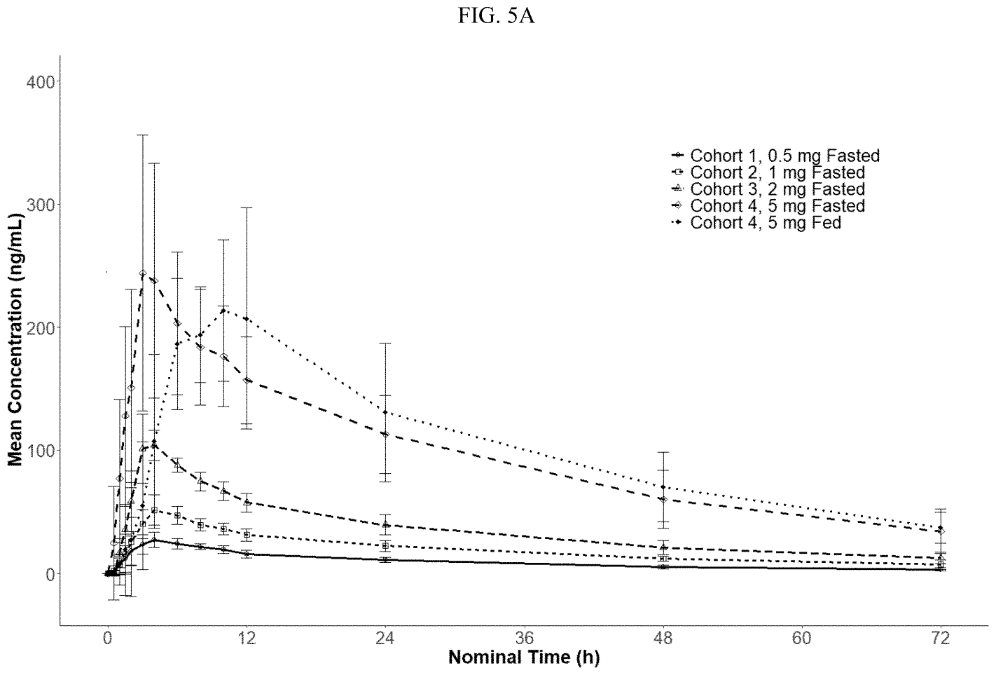

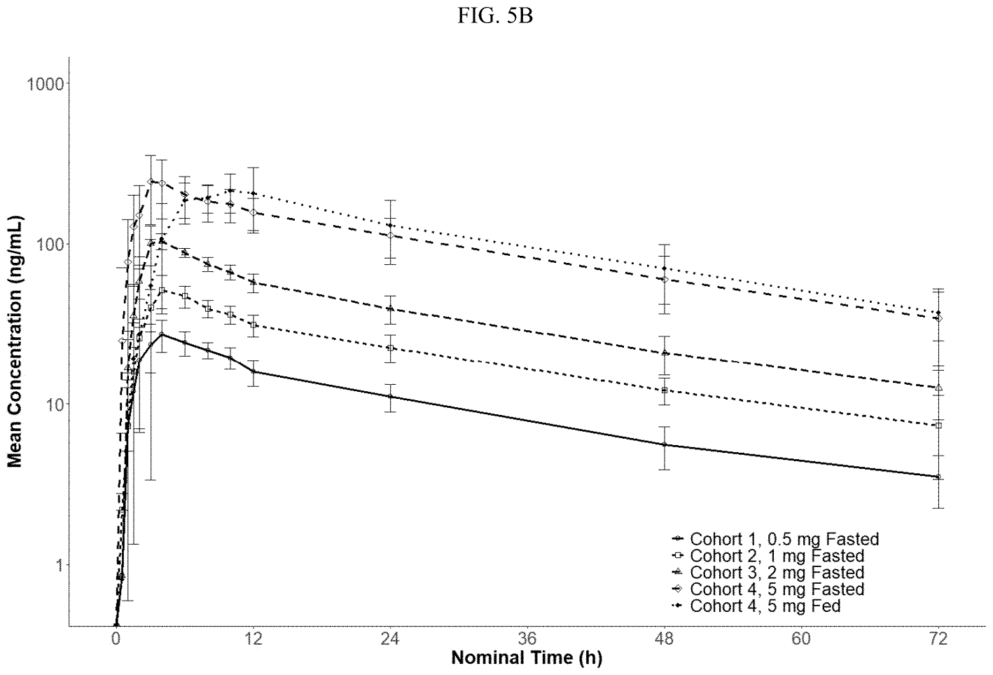

FIG. 5A and FIG. 5B show mean plasma concentration of Reference Example 1 compound following single dose of 0.5 mg (fasted), 1 mg (fasted), 2 mg (fasted), 5 mg (fasted), and 5 mg (fed) in linear scale and semi-logarithmic scale, respectively. Analysis of the effects of food on Reference Example 1 compound showed a minimal increase in exposure following when the compound was administered with food. Fed/fasted % ratios for Cₘₐₓ and AUCₗₐₛₜ were 103.19 and 106.28, respectively, indicating no relevant effect when the compound was administered with food.

Regarding the safety, 12 adverse event (AE) were reported, but no adverse event of special interest (AESI) or serious adverse event (SAE) was reported. Of the 12 AEs, 5 were considered to be possibly related to IMP (Investigational Medicinal Product). The 5 AE included headaches (2), lower abdominal pain, diarrhea, and exanthema in the right upper arm, all of which were of mild intensity and recovered quickly.

Overall, none of the AEs caused medical concern, and no stopping criteria per protocol were met. For all subjects, no significant changes were recorded in ECG, vital signs, and lab data. According to the safety assessments obtained in the study it can be concluded that up to single dose of 10 mg Reference Example 1 compound or placebo was safe and well tolerated in all subjects.

### Example 11: Clinical Study (Multiple Ascending Dose (MAD) Study in Healthy Human)

To further study safety and pK data in human subjects, multiple ascending dose (MAD) study is conducted. MAD study include 3 cohorts of 10 healthy subjects per cohort, who are randomized and received an oral dose of Reference Example 1 compound or placebo (4:1) ranging from 1 to 5 mg for 14 days, according to the safety results from each cohort as determined from the results of SAD study.

A population of PK model was developed based on the single dose PK data under fasting condition, described in Example 10. The model structure was a two-compartment model with linear elimination and two subsequent first-order absorption. The Model-predicted exposure on Day 14 (steady-state) after oral daily dosing are shown in Table 3.

TABLE 3
Model-predicted human Reference Example 1 compound
exposure at selected multiple ascending doses

| | Predicted steady-state exposure | | Safety margin |
|---|---|---|---|
| | Median of | | Median of |
| 14 Day Dose | AUC_{24 hr} | Median of | *BSEP |
| (mg) | (h*ng/mL) | Cₘₐₓ (ng/mL) | IC₅₀/Cₘₐₓ |
| 1 | 1,627 | 95 | 75 |
| 2.5 | 4,067 | 238 | 30 |
| 5 | 8,134 | 476 | 15 |

*BSEP (bile salt export pump) IC₅₀ of Reference Example 1 compound = 7,172 ng/mL

The model-predicted safety margin of 15 at 5 mg dose (for 14 days) is greater than safety margin criteria of 10, indicating that the Reference Example 1 compound is safe.

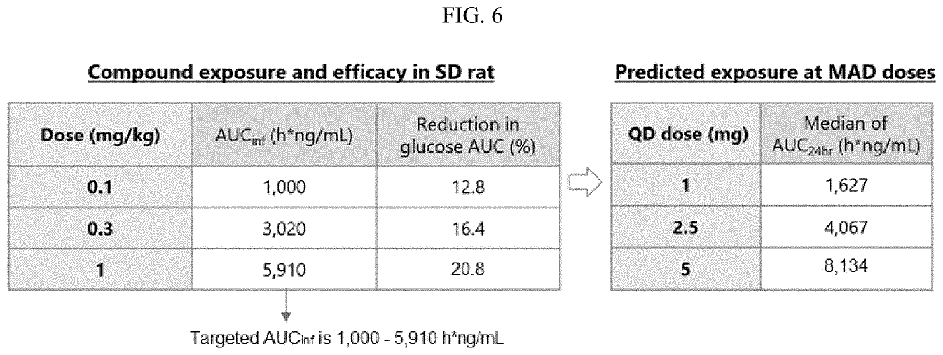

Additionally, the results of the exposure and efficacy of Reference Example 1 compound (at 0.1 mg/kg, 0.3 mg/kg, and 1 mg/kg doses) in SD rats are shown in FIG. 6. The lowest MAD dose 1 mg in human could provide exposure higher than Minimum Effective Exposure in SD rats, and the highest 5 mg could cover the exposure in SD rats at 1 mg/kg that could produce approximately 20.8% reduction in glucose AUC in OGTT (Oral Glucose Tolerance Test).

### Example 12: Drug-Induced Liver Injury (DILI) Assessment

A human clinical study of Fasiglifam, a GPR40 agonist, was terminated due to potential liver toxicity. Reference Example 1 compound showed a greatly superior safety profile in drug-induced liver injury (DILI) study.

DILI risk may be due to potency of BSEP inhibition in combination with in vivo drug exposure (e.g., Cₘₐₓ or Cₛₛ (plasma concentration at steady state)). Majority of drug with DILI risk show the safety margin of 10 or less, in which the safety margin is calculated by dividing IC₅₀ by Cₘₐₓ or Cₛₛ. Table 4 shows the safety margin reported for fasiglifam and calculated for Reference Example 1 compound.

TABLE 4
BSEP IC₅₀ and Safety Margin of Fasiglifam and Ref Ex 1 Compound

| Parameter | fasiglifam | Ref Ex 1 Compd |
|---|---|---|
| Human Cₘₐₓ (μM) | 10.1¹⁾ | 0.3²⁾ |
| BSEP IC₅₀ (μM) | 19.6 | 14.3 |
| Safety margin | 1.9 | 42.1 |
| (IC₅₀/Cₘₐₓ) | | |

Cₘₐₓ (Maximum plasma concentration at steady state)
¹⁾Clin. Pharmacol. Ther. 92, 29-39.
²⁾Estimated Cₘₐₓ value derived from PK/PD modeling (Example 1).

(a) Human Transporter Inhibition Study and Safety Margin

Fasiglifam and Reference Example 1 compound were tested for determining in vitro inhibition IC50 (μM) on various drug transporters including BSEP, MRP2, MRP3, and MRP4, and determined the safety margins for these drug transporters. The results are showsn in Table 5. The results of Table 5 show that Reference Example 1 compound has a higher safety margin on BSEP, MRP2, 3 and 4 inhibition compared to fasiglifam and has a lower risk of DILI.

TABLE 5
Drug Transports IC₅₀ and Safety Margin of Fasiglifam
and Ref Ex 1 Compound

| | In vitro Inhibition IC₅₀ (μM) | | | |
|---|---|---|---|---|
| Drug transporter | fasiglifam | | Ref. Ex. 1 Compound | |
| BSEP | 19.6 | | 14.3 | |
| MRP2 | 24.8 | | 39.9 | |
| MRP3 | 14.2 | | No inhibition | |
| MRP4 | 11.1 | | 4.4 | |
| | Safety margin (IC₅₀/C_{pss, max}) | | | |
| | fasiglifam | | Ref. Ex. 1 Compound | |
| Dose (mg) | 25 | 50 | 1 | 2 |
| C_{pss, max} (μM) | 4.38 | 10.10 | 0.17 | 0.34 |
| BSEP | 4.5 | 1.9 | 84.1 | 42.1 |
| MRP2 | 5.7 | 2.5 | 234.7 | 117.4 |
| MRP3 | 3.2 | 1.4 | No inhibition | |
| MRP4 | 2.5 | 1.1 | 25.9 | 13.0 |

(b) Bile Acid (BA) Analysis—Glycocholic Acid (GCA) Accumulation

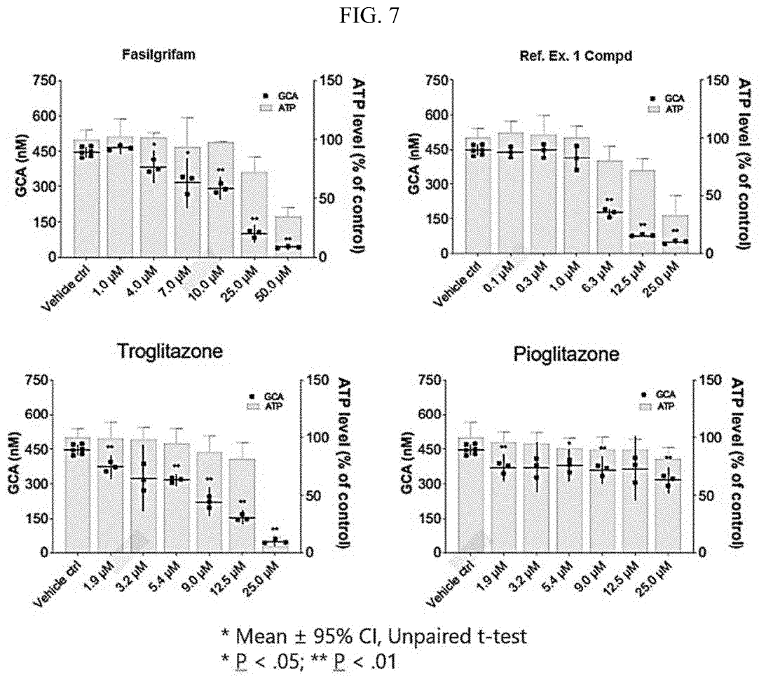

Glycocholic acid (GCA) and glycochenodeoxycholic acid (GCDCA) are major components of human bile acids and their significant increase has been confirmed in DILI patients. Fasiglifam, Troglitazone, Pioglitazone, and Reference Example 1 compound at various concentrations were tested for accumulation of GCA. The results are shown in FIG. 7.

Fasiglifam induces significant accumulation of GCA at 4 μM below the human Cmax of 10 μM, whereas Reference Example 1 compound does not show significant accumulation at 1 μM above the expected human Cₘₐₓ of 0.3 μM.
(c) Mitochondrial Function Inhibition

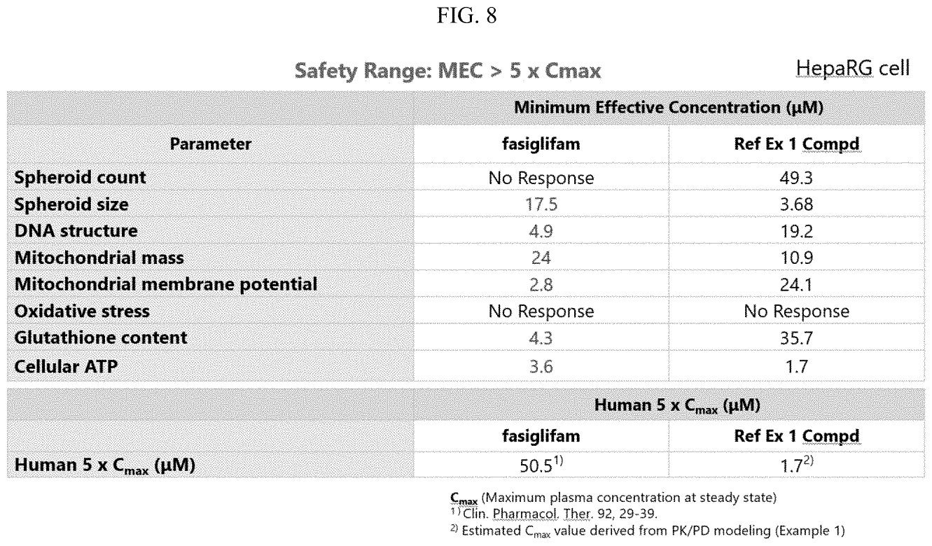

The inhibitory effects of fasiglifam and Reference Example 1 compound on mitochondrial function were evaluated using HepaRG cell. The results are shown in FIG. 8. Reference Example 1 compound shows lower DILI risk than fasiglifam on mitochondria in vitro assay.
(d) Covalent Protein Binding in Human Hepatocytes

The covalent binding (CVB) burden was estimated by determining the CVB of radiolabeled compound to human hepatocytes and factoring in both the daily dose and the fraction of metabolism leading to CVB. The CVB burden of Reference Example 1 compound using the clinical dose of 2 mg (based on Examples 1 and 10) was 0.01 mg/day, which was notably lower than fasiglifam's 2 mg/day. Reference Example 1 compound's dose over 260 mg a day was predicted to exceed the CVB burden threshold of 1 mg/day. The results are shown in Table 6.

TABLE 6
Covalent Protein Binding in Human Hepatocytes

| | | Covalent binding (CVB, pmol/mg protein) | | | CVB |
|---|---|---|---|---|---|
| | Daily dose | | +Aminobenzotriazole | +Borneol | Burden ²⁾ |
| Compound | (mg) | Compound | (CYP inhibitor) | (UGT inhibitor) | (mg/day) |
| Threshold to | | >50 ¹⁾ | | | >1 ²⁾ |
| consider DILI risk | | | | | |
| C¹⁴-fasiglifam ³⁾ | 50 | 69.1 | 73.7 | 44.4 | 2 |
| C¹⁴-Ref. Ex. 1. C. | 2 ⁴⁾ | 37.6 | 29.5 | 21.2 | 0.01 |

¹⁾ Toru Usui et al., Drug Metabolism & Disposition, 37: 2383-2392, 2009.
²⁾ Drugs with a CVB >1 mg/day are associated with a high risk for DILI (Thompson et al., Research in Toxicology 2012).
³⁾ Otieno et al., TOXICOLOGICAL SCIENCES, 163(2): 374-384, 2018.
⁴⁾ Human efficacious dose (2 mg) was obtained through PK/PD modeling (Example 1)

(e) HμRELTox™ Assay

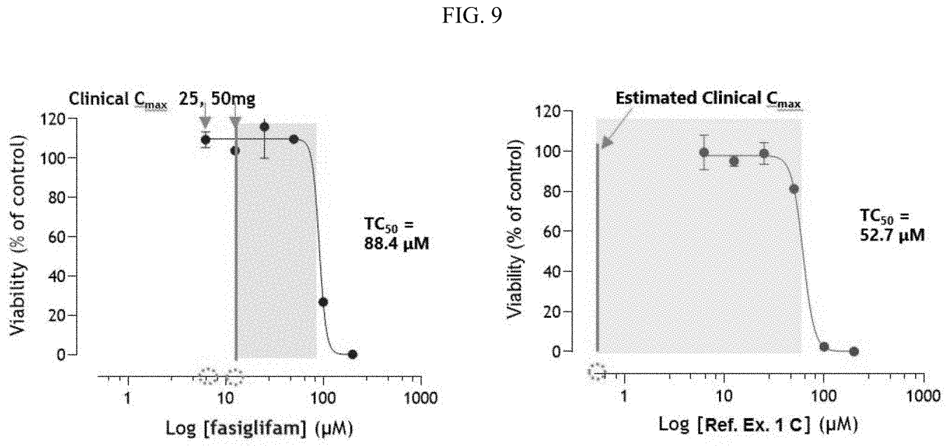

Fasiglifam and Reference Example 1 compound were tested for hepatotoxic responses using HμRELTOX™ assay, and results are shown in Table 7 and FIG. 9. The results show that Reference Example 1 compound and fasiglifam exhibited a similar level of hepatotoxic response TC₅₀), and Reference Example 1 compound has a broader safety range than fasiglifam.

TABLE 7
HμRELTOX ™ Assay

| | fasiglifam | Ref. Ex. 1 Compound |
|---|---|---|
| Human Cₘₐₓ (μM) | 10.1¹⁾ | 0.3²⁾ |
| TC₅₀ (μM) | 88.4 | 52.7 |
| Safety margin | 8.8 | 156.8 |
| (TC₅₀/Cₘₐₓ) | | |

Cₘₐₓ (Maximum plasma concentration at steady-state)
¹⁾Clin. Pharmacol. Ther. 92, 29-39.
²⁾Estimated Cₘₐₓ value derived from PK/PD modeling (Example 1)

(f) Transcription Factor (TF) Profiling

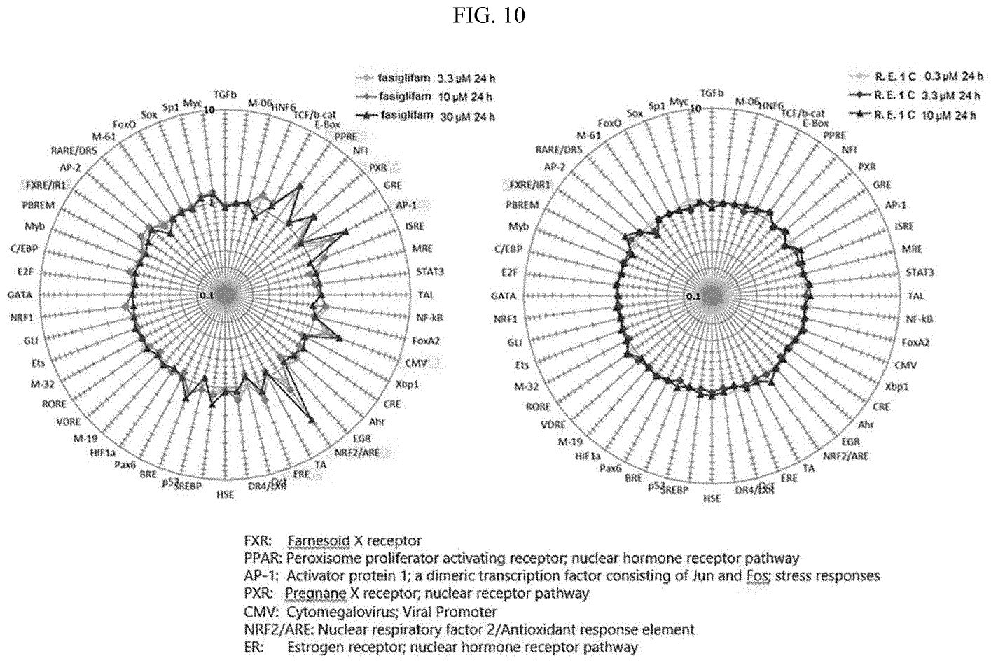

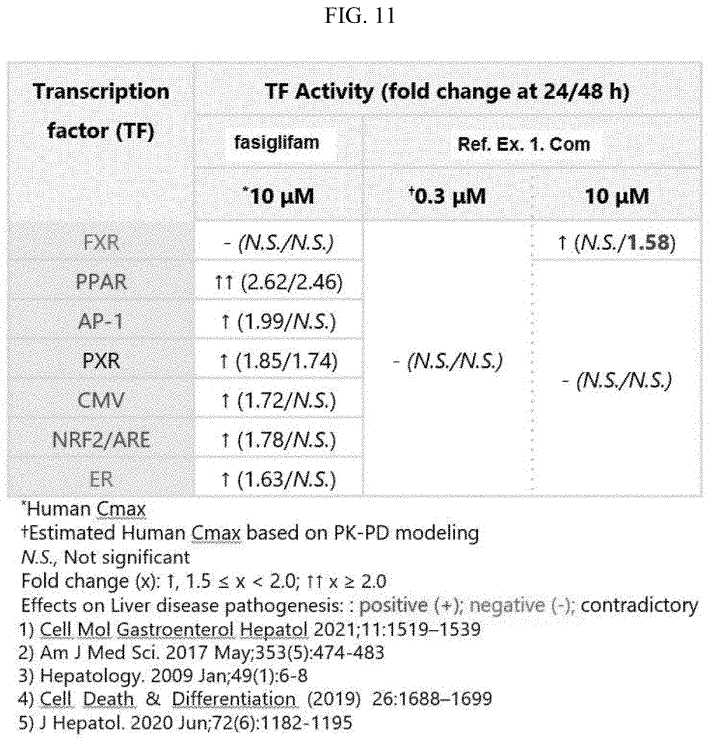

Effects of fasiglifam and Reference Example 1 compound on various hepatic transcription factors were assayed using 2D HepG2 cell by treating the cell with 3.3 μM, 10 μM, and 30 μM of fasiglifam for 24 hours or 0.3 μM, 3.3 μM, and 10 μM of Reference Example 1 compound for 24 hours. The results are shown in FIGS. 10 and 11.

The effect of Reference Example 1 compound on quantitative evaluations of various transcription factor (TF) activities is insignificant compared to that of fasiglifam. Treatment with 10 μM Reference Example 1 compound for 48 hours increased the activity of FXR, a master regulator of BA metabolism, by 1.58-fold. Treatment with 10 μM fasiglifam for 24 hours significantly increased the activity of PPAR, AP-1, and NRF2, which are highly correlated with liver disease pathogenesis/progression.

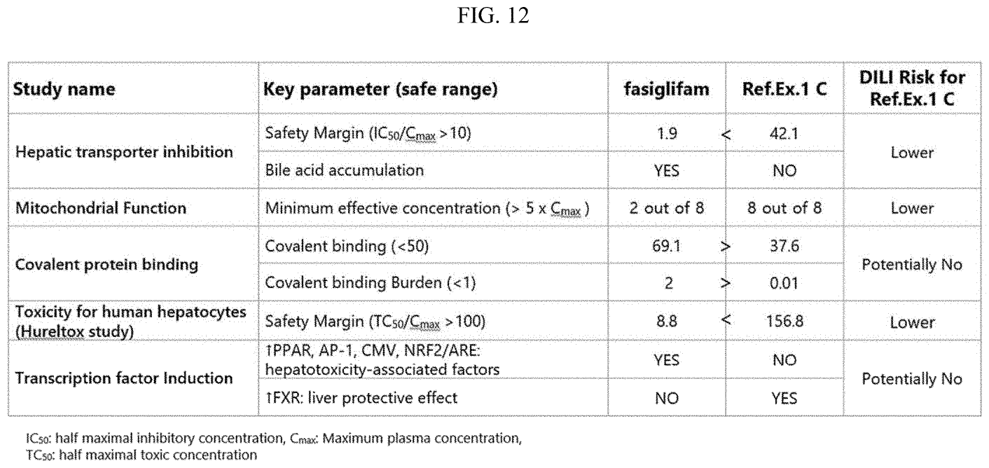

FIG. 12 summarizes the DILI Assessment comparing the Reference Example 1 compound and fasiglifam. The summary of FIG. 12 clearly shows that the compounds of Formula (I) including Reference Example 1 compound are safe and show a significantly lower DILI risk compared to fasiglifam.

The specific pharmacological and biochemical responses observed in the experiments described may vary according to and depending on whether there are present pharmaceutical carriers as well as type of formulation and mode of administration employed, and such expected variations or differences in the results are contemplated in accordance with practice of the embodiments of the invention.

1. A method for treating a subject with diabetes or pre-diabetes, comprising administering to the subject an effective amount of a phenyl propionic acid of the following Formula (I), an isomer, or a pharmaceutically acceptable salt thereof: 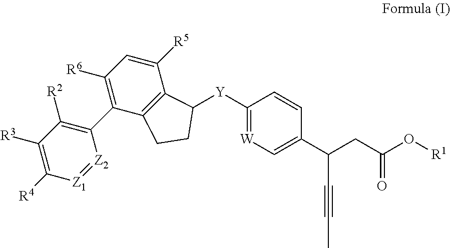
R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ and R⁴ are each independently hydrogen, halogen, cyano, C₁₋₄ linear or branched alkoxy, or OR⁸;
wherein R⁸ is hydrogen, C₃₋₁₀ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ linear or branched alkoxy;
Y is NH or O;
Z₁, Z₂ and W are each independently CR² or N;
wherein R⁷ is hydrogen, halogen, cyano, hydroxyl, C₁₋₄ linear or branched alkyl, or C₁₋₄ linear or branched alkoxy,
wherein the administering of the compound of Formula (I), an isomer, or a pharmaceutically acceptable salt thereof to the subject lowers one or more of HbA1c level, fasting plasma glucose level, 2-hour oral glucose tolerance test (OGTT) result level, and random plasma glucose level.

2. The method of claim 1, wherein the diabetes is type 2 diabetes.

3. The method of claim 1, wherein the compound of Formula (I) is
(1) (S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid;
(2) (S)-3-(4-(((R)-7-fluoro-4-(6-((3-methyloxetan-3-yl)methoxy)pyridin-3-yl)-2,3-dihydro-1H-inden-1-yl)oxy)phenyl)hex-4-ynoic acid;
(3) (S)-3-(4-(((R)-4-(6-(2-(1,1-dioxidothiomorpholino)ethoxy)pyridin-3-yl)-7-fluoro-2,3-dihydro-1H-inden-1-yl)oxy)phenyl)hex-4-ynoic acid;
(4) (S)-3-(4-(((R)-7-fluoro-4-(6-(oxetan-3-yloxy)pyridin-3-yl)-2,3-dihydro-1H-inden-1-yl)oxy)phenyl)hex-4-ynoic acid;
(5) (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid;
(6) (S)-3-(4-(((R)-7-fluoro-4-(6-((tetrahydro-2H-pyran-4-yl)oxy)pyridin-3-yl)-2,3-dihydro-1H-inden-1-yl)oxy)phenyl)hex-4-ynoic acid;
(7) (S)-3-(4-(((R)-7-fluoro-4-(6-(((S)-tetrahydrofuran-3-yl)oxy)pyridin-3-yl)-2,3-dihydro-1H-inden-1-yl)oxy)phenyl)hex-4-ynoic acid;
(8) (S)-3-(4-(((R)-7-fluoro-4-(4-methyl-6-(((R)-tetrahydrofuran-3-yl)oxy)pyridin-3-yl)-2,3-dihydro-1H-inden-1-yl)oxy)phenyl)hex-4-ynoic acid;
(9) (S)-3-(4-(((R)-7-fluoro-4-(2-methyl-6-(((R)-tetrahydrofuran-3-yl)oxy)pyridin-3-yl)-2,3-dihydro-1H-inden-1-yl)oxy)phenyl)hex-4-ynoic acid;
(10) (S)-3-(4-(((R)-4-(5-chloro-6-(((R)-tetrahydrofuran-3-yl)oxy)pyridin-3-yl)-7-fluoro-2,3-dihydro-1H-inden-1-yl)oxy)phenyl)hex-4-ynoic acid;
(11) (S)-3-(4-(((R)-7-fluoro-4-(5-(((R)-tetrahydrofuran-3-yl)oxy)pyridin-2-yl)-2,3-dihydro-1H-inden-1-yl)oxy)phenyl)hex-4-ynoic acid;
(12) (S)-3-(4-(((R)-7-fluoro-4-(4-methyl-6-((3-methyloxetan-3-yl)methoxy)pyridin-3-yl)-2,3-dihydro-1H-inden-1-yl)oxy)phenyl)hex-4-ynoic acid;
(13) (S)-3-(4-(((R)-7-fluoro-4-(2-methyl-6-((3-methyloxetan-3-yl)methoxy)pyridin-3-yl)-2,3-dihydro-1H-inden-1-yl)oxy)phenyl)hex-4-ynoic acid;
(14) (S)-3-(4-(((R)-7-fluoro-4-(5-((3-methyloxetan-3-yl)methoxy)pyridin-2-yl)-2,3-dihydro-1H-inden-1-yl)oxy)phenyl)hex-4-ynoic acid;
(15) (S)-3-(4-(((R)-7-fluoro-4-(5-(((R)-tetrahydrofuran-3-yl)oxy)pyridin-3-yl)-2,3-dihydro-1H-inden-1-yl)oxy)phenyl)hex-4-ynoic acid;
(16) (S)-3-(4-(((R)-7-fluoro-4-(5-((tetrahydro-2H-pyran-4-yl)oxy)pyridin-3-yl)-2,3-dihydro-1H-inden-1-yl)oxy)phenyl)hex-4-ynoic acid;
(17) (S)-3-(4-(((R)-4-(5-chloro-6-((tetahydro-2H-pyran-4-yl)oxy)pyridin-3-yl)-7-fluoro-2,3-dihydro-1H-inden-1-yl)oxy)phenyl)hex-4-ynoic acid;
(18) (S)-3-(4-(((R)-4-(5-cyano-6-(((R)-tetrahydrofuran-3-yl)oxy)pyridin-3-yl)-7-fluoro-2,3-dihydro-1H-inden-1-yl)oxy)phenyl)hex-4-ynoic acid;
(19) (S)-3-(4-(((R)-4-(5-cyano-6-((tetahydro-2H-pyran-4-yl)oxy)pyridin-3-yl)-7-fluoro-2,3-dihydro-1H-inden-1-yl)oxy)phenyl)hex-4-ynoic acid;
(20) (S)-3-(4-(((R)-5-cyano-4-(6-(((R)-tetrahydrofuran-3-yl)oxy)pyridin-3-yl)-2,3-dihydro-1H-inden-1-yl)oxy)phenyl)hex-4-ynoic acid;
(21) (S)-3-(4-(((R)-5-fluoro-4-(6-(((R)-tetrahydrofuran-3-yl)oxy)pyridin-3-yl)-2,3-dihydro-1H-inden-1-yl)oxy)phenyl)hex-4-ynoic acid;
(22) (S)-3-(4-(((R)-5-methoxy-4-(6-(((R)-tetrahydrofuran-3-yl)oxy)pyridin-3-yl)-2,3-dihydro-1H-inden-1-yl)oxy)phenyl)hex-4-ynoic acid;
(23) (S)-3-(4-(((R)-5-cyano-4-(6-((tetrahydro-2H-pyran-4-yl)oxy)pyridin-3-yl)-2,3-dihydro-1H-inden-1-yl)oxy)phenyl)hex-4-ynoic acid;
(24) (S)-3-(4-(((R)-5-fluoro-4-(6-((tetrahydro-2H-pyran-4-yl)oxy)pyridin-3-yl)-2,3-dihydro-1H-inden-1-yl)oxy)phenyl)hex-4-ynoic acid;
(25) (S)-3-(4-(((R)-5-methoxy-4-(6-((tetrahydro-2H-pyran-4-yl)oxy)pyridin-3-yl)-2,3-dihydro-1H-inden-1-yl)oxy)phenyl)hex-4-ynoic acid;
(26) (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid; or
(27) 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid.

4. A method for preventing and/or treating type 2 diabetes in a subject in need thereof, comprising administering an effective amount of (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid, an isomer, or a pharmaceutically acceptable salt thereof to the subject.

5. The method of claim 1, wherein the effective amount is about 0.5 mg to 30 mg/day.

6. The method of claim 1, wherein the compound of Formula (I), an isomer, or a pharmaceutically acceptable salt is administered orally.

7. The method of claim 1, which further comprises administering one or more antidiabetic agents.

8. The method of claim 7, wherein the one or more antidiabetic agents are selected from the group consisting of biguanide, a dipeptidyl peptidase-4 (DPP-4) inhibitor, a sulphonylurea, a thiazolidinedione, a meglitinide, an alpha-glucosidase blocker, a glucagon-like peptide-1 receptor agonist, insulin, and an insulin analog.

9. A method for treating a subject with metabolic disease, comprising administering to the subject an effective amount of a phenyl propionic acid of the following Formula (I), an isomer, or a pharmaceutically acceptable salt thereof: 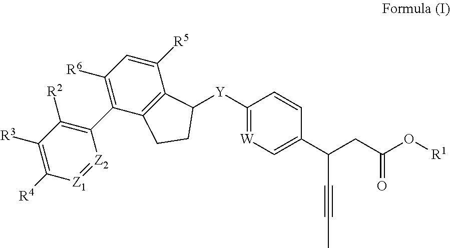
R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ and R⁴ are each independently hydrogen, halogen, cyano, C₁₋₄ linear or branched alkoxy, or OR⁸;
wherein R⁸ is hydrogen, C₃₋₁₀ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ linear or branched alkoxy;
Y is NH or O;
Z₁, Z₂ and W are each independently CR⁷ or N;
wherein R⁷ is hydrogen, halogen, cyano, hydroxyl, C₁₋₄ linear or branched alkyl, or C₁₋₄ linear or branched alkoxy,
wherein the administering of the compound of Formula (I), an isomer, or a pharmaceutically acceptable salt thereof to the subject lowers one or more of HbA1c level, fasting plasma glucose level, 2-hour oral glucose tolerance test (OGTT) result level, and random plasma glucose level.

10. The method of claim 9, wherein the subject shows one, two or more of the following conditions:
(a) a fasting blood glucose or serum glucose concentration greater than 100 mg/dL or greater than 110 mg/dL, in particular greater than 125 mg/dL;
(b) a postprandial plasma glucose equal to or greater than 140 mg/dL;
(c) an HbA1c value equal to or greater than 5.7%, equal to or greater than 6.5%, equal to or greater than 7.0%, equal to or greater than 7.5%, or equal to or greater than 8.0%.

11. The method of claim 9, wherein the compound of Formula (I) is
(1) (S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid;
(2) (S)-3-(4-(((R)-7-fluoro-4-(6-((3-methyloxetan-3-yl)methoxy)pyridin-3-yl)-2,3-dihydro-1H-inden-1-yl)oxy)phenyl)hex-4-ynoic acid;
(3) (S)-3-(4-(((R)-4-(6-(2-(1,1-dioxidothiomorpholino)ethoxy)pyridin-3-yl)-7-fluoro-2,3-dihydro-1H-inden-1-yl)oxy)phenyl)hex-4-ynoic acid;
(4) (S)-3-(4-(((R)-7-fluoro-4-(6-(oxetan-3-yloxy)pyridin-3-yl)-2,3-dihydro-1H-inden-1-yl)oxy)phenyl)hex-4-ynoic acid;
(5) (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid;
(6) (S)-3-(4-(((R)-7-fluoro-4-(6-((tetrahydro-2H-pyran-4-yl)oxy)pyridin-3-yl)-2,3-dihydro-1H-inden-1-yl)oxy)phenyl)hex-4-ynoic acid;
(7) (S)-3-(4-(((R)-7-fluoro-4-(6-(((S)-tetrahydrofuran-3-yl)oxy)pyridin-3-yl)-2,3-dihydro-1H-inden-1-yl)oxy)phenyl)hex-4-ynoic acid;
(8) (S)-3-(4-(((R)-7-fluoro-4-(4-methyl-6-(((R)-tetrahydrofuran-3-yl)oxy)pyridin-3-yl)-2,3-dihydro-1H-inden-1-yl)oxy)phenyl)hex-4-ynoic acid;
(9) (S)-3-(4-(((R)-7-fluoro-4-(2-methyl-6-(((R)-tetrahydrofuran-3-yl)oxy)pyridin-3-yl)-2,3-dihydro-1H-inden-1-yl)oxy)phenyl)hex-4-ynoic acid;
(10) (S)-3-(4-(((R)-4-(5-chloro-6-(((R)-tetrahydrofuran-3-yl)oxy)pyridin-3-yl)-7-fluoro-2,3-dihydro-1H-inden-1-yl)oxy)phenyl)hex-4-ynoic acid;
(11) (S)-3-(4-(((R)-7-fluoro-4-(5-(((R)-tetrahydrofuran-3-yl)oxy)pyridin-2-yl)-2,3-dihydro-1H-inden-1-yl)oxy)phenyl)hex-4-ynoic acid;
(12) (S)-3-(4-(((R)-7-fluoro-4-(4-methyl-6-((3-methyloxetan-3-yl)methoxy)pyridin-3-yl)-2,3-dihydro-1H-inden-1-yl)oxy)phenyl)hex-4-ynoic acid;
(13) (S)-3-(4-(((R)-7-fluoro-4-(2-methyl-6-((3-methyloxetan-3-yl)methoxy)pyridin-3-yl)-2,3-dihydro-1H-inden-1-yl)oxy)phenyl)hex-4-ynoic acid;
(14) (S)-3-(4-(((R)-7-fluoro-4-(5-((3-methyloxetan-3-yl)methoxy)pyridin-2-yl)-2,3-dihydro-1H-inden-1-yl)oxy)phenyl)hex-4-ynoic acid;
(15) (S)-3-(4-(((R)-7-fluoro-4-(5-(((R)-tetrahydrofuran-3-yl)oxy)pyridin-3-yl)-2,3-dihydro-1H-inden-1-yl)oxy)phenyl)hex-4-ynoic acid;
(16) (S)-3-(4-(((R)-7-fluoro-4-(5-((tetrahydro-2H-pyran-4-yl)oxy)pyridin-3-yl)-2,3-dihydro-1H-inden-1-yl)oxy)phenyl)hex-4-ynoic acid;
(17) (S)-3-(4-(((R)-4-(5-chloro-6-((tetrahydro-2H-pyran-4-yl)oxy)pyridin-3-yl)-7-fluoro-2,3-dihydro-1H-inden-1-yl)oxy)phenyl)hex-4-ynoic acid;
(18) (S)-3-(4-(((R)-4-(5-cyano-6-(((R)-tetrahydrofuran-3-yl)oxy)pyridin-3-yl)-7-fluoro-2,3-dihydro-1H-inden-1-yl)oxy)phenyl)hex-4-ynoic acid;
(19) (S)-3-(4-(((R)-4-(5-cyano-6-((tetrahydro-2H-pyran-4-yl)oxy)pyridin-3-yl)-7-fluoro-2,3-dihydro-1H-inden-1-yl)oxy)phenyl)hex-4-ynoic acid;
(20) (S)-3-(4-(((R)-5-cyano-4-(6-(((R)-tetrahydrofuran-3-yl)oxy)pyridin-3-yl)-2,3-dihydro-1H-inden-1-yl)oxy)phenyl)hex-4-ynoic acid;
(21) (S)-3-(4-(((R)-5-fluoro-4-(6-(((R)-tetrahydrofuran-3-yl)oxy)pyridin-3-yl)-2,3-dihydro-1H-inden-1-yl)oxy)phenyl)hex-4-ynoic acid;
(22) (S)-3-(4-(((R)-5-methoxy-4-(6-(((R)-tetrahydrofuran-3-yl)oxy)pyridin-3-yl)-2,3-dihydro-1H-inden-1-yl)oxy)phenyl)hex-4-ynoic acid;
(23) (S)-3-(4-(((R)-5-cyano-4-(6-((tetmhydro-2H-pyran-4-yl)oxy)pyridin-3-yl)-2,3-dihydro-1H-inden-1-yl)oxy)phenyl)hex-4-ynoic acid;
(24) (S)-3-(4-(((R)-5-fluoro-4-(6-((tetrahydro-2H-pyran-4-yl)oxy)pyridin-3-yl)-2,3-dihydro-1H-inden-1-yl)oxy)phenyl)hex-4-ynoic acid;
(25) (S)-3-(4-(((R)-5-methoxy-4-(6-((tetrahydro-2H-pyran-4-yl)oxy)pyridin-3-yl)-2,3-dihydro-1H-inden-1-yl)oxy)phenyl)hex-4-ynoic acid;
(26) (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid; or
(27) 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid.

12. The method of claim 9, wherein the compound of Formula (I) is (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid.

13. The method of claim 9, wherein the effective amount is about 0.5 mg to about 30 mg/day.

14. The method of claim 9, wherein the compound of Formula (I), an isomer, or a pharmaceutically acceptable salt is administered orally.

15. The method of claim 9, which further comprises administering one or more antidiabetic agents.

16. The method of claim 15, wherein the one or more antidiabetic agents are selected from the group consisting of biguanide, a dipeptidyl peptidase-4 (DPP-4) inhibitor, a sulphonylurea, a thiazolidinedione, a meglitinide, an alpha-glucosidase blocker, a glucagon-like peptide-1 receptor agonist, insulin, and an insulin analog.

17. A method selected from the group consisting of
(i) improving glycemic control and/or for reducing of fasting plasma glucose and/or of postprandial plasma glucose and/or of glycosylated hemoglobin HbA1c,
(ii) preventing, slowing the progression of, delaying, or treating a metabolic disorder selected from the group consisting of type 1 diabetes, type 2 diabetes, impaired glucose tolerance, impaired fasting blood glucose, hyperglycemia, postprandial hyperglycemia, overweight, obesity, and metabolic syndrome,
(iii) preventing, slowing, delaying, or reversing progression from impaired glucose tolerance, insulin resistance, and/or from metabolic syndrome to type 2 diabetes mellitus,
(iv) preventing, slowing the progression of, delaying, or treating of a condition or disorder selected from the group consisting of cataracts, nephropathy, retinopathy, neuropathy, learning and memory impairment, neurodegenerative or cognitive disorders, cardio- or cerebrovascular diseases, tissue ischemia, diabetic foot ulcer, arteriosclerosis, hypertension, endothelial dysfunction, myocardial infarction, acute coronary syndrome, unstable angina pectoris, stable angina pectoris, stroke, peripheral arterial occlusive disease, cardiomyopathy, heart failure, heart rhythm disorders, and vascular restenosis,
(v) preventing, slowing, delaying, or treating the degeneration of pancreatic beta cells and/or the decline of the functionality of pancreatic beta cells and/or for improving and/or restoring or protecting the functionality of pancreatic beta cells and/or restoring the functionality of pancreatic insulin secretion,
(vi) preventing, slowing, delaying or treating diseases or conditions attributed to an abnormal accumulation of liver or ectopic fat, and
(vii) maintaining and/or improving the insulin sensitivity and/or for treating or preventing hyperinsulinemia and/or insulin resistance,
the method comprising administering to a subject in need thereof an effective amount of:
(a) a compound of the following Formula (I), an isomer, or a pharmaceutically acceptable salt thereof,
(b) optionally, a second hypoglycemic agent selected from the group consisting of biguanides, thiazolidinediones, sulfonylureas, glinides, alpha-glucosidase blockers, GLP-1 and GLP-1 analogues, or a pharmaceutically acceptable salt thereof, and,
(c) optionally, a third hypoglycemic agent different from (b) and selected from the group consisting of biguanides, thiazolidinediones, sulfonylureas, glinides, alpha-glucosidase blockers, GLP-1 and GLP-1 analogues, or a pharmaceutically acceptable salt thereof,
wherein the subject shows one, two or more of the following conditions:
(A) a fasting blood glucose or serum glucose concentration greater than 100 mg/dL or greater than 110 mg/dL, in particular greater than 125 mg/dL;
(B) a postprandial plasma glucose equal to or greater than 140 mg/dL;
(C) an HbA1c value equal to or greater than 5.7%, equal to or greater than 6.5%, equal to or greater than 7.0%, equal to or greater than 7.5%, or equal to or greater than 8.0%, 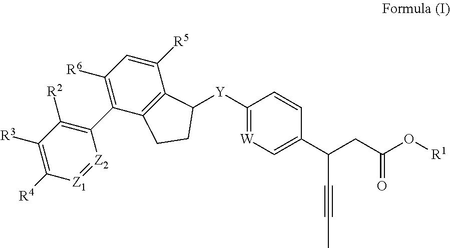
R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ and R⁴ are each independently hydrogen, halogen, cyano, C₁₋₄ linear or branched alkoxy, or OR⁸;
wherein R⁸ is hydrogen, C₃₋₄ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ linear or branched alkoxy;
Y is NH or O;
Z₁, Z₂ and W are each independently CR⁷ or N;
wherein R⁷ is hydrogen, halogen, cyano, hydroxyl, C₁₋₄ linear or branched alkyl, or C₁₋₄ linear or branched alkoxy.

18. The method of claim 5, wherein the effective amount is about 1 mg to about 5 mg/day, about 5 mg to about 10 mg/day, about 10 mg to about 15 mg/day, about 15 mg to about 20 mg/day, about 20 mg to about 25 mg/day, about 25 mg to about 30 mg/day, about 1 mg/day, about 2 mg/day, about 3 mg/day, about 4 mg/day, about 5 mg/day, about 6 mg/day, about 7 mg/day, about 7.5 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 21 mg/day, about 22 mg/day, about 23 mg/day, about 24 mg/day, about 25 mg/day, about 26 mg/day, about 27 mg/day, about 28 mg/day, about 29 mg/day, or about 30 mg/day.

19. The method of claim 1, wherein the compound of Formula (I), an isomer, or a pharmaceutically acceptable salt is administered once a day.

20. The method of claim 13, wherein the effective amount is about 1 mg to about 5 mg/day, about 5 mg to about 10 mg/day, about 10 mg to about 15 mg/day, about 15 mg to about 20 mg/day, about 20 mg to about 25 mg/day, about 25 mg to about 30 mg/day, about 1 mg/day, about 2 mg/day, about 3 mg/day, about 4 mg/day, about 5 mg/day, about 6 mg/day, about 7 mg/day, about 7.5 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 21 mg/day, about 22 mg/day, about 23 mg/day, about 24 mg/day, about 25 mg/day, about 26 mg/day, about 27 mg/day, about 28 mg/day, about 29 mg/day, or about 30 mg/day.

21. The method of claim 9, wherein the compound of Formula (I), an isomer, or a pharmaceutically acceptable salt is administered once a day.
